SEQUENCE LISTING (192 sequences)

SEQ ID NO: 1 - DNA, 20 bp, synthetic construct
  misc_feature 1..20: note = Synthetic oligonucleotide
  source 1..20: mol_type = other DNA
aacaagagcctgggacctcc

SEQ ID NO: 2 - DNA, 20 bp, synthetic construct
  misc_feature 1..20: note = Synthetic oligonucleotide
  source 1..20: mol_type = other DNA
gcagaaggtgaaggggagca

SEQ ID NO: 3 - DNA, 21 bp, synthetic construct
  misc_feature 1..21: note = Synthetic oligonucleotide
  source 1..21: mol_type = other DNA
agctctgccctcatgaatgct

SEQ ID NO: 4 - DNA, 20 bp, synthetic construct
  misc_feature 1..20: note = Synthetic oligonucleotide
  source 1..20: mol_type = other DNA
tcatgaaccgcctctcacga

SEQ ID NO: 5 - DNA, 20 bp, synthetic construct
  misc_feature 1..20: note = Synthetic oligonucleotide
  source 1..20: mol_type = other DNA
aatggcgtgaacccgggaag

SEQ ID NO: 6 - DNA, 19 bp, synthetic construct
  misc_feature 1..19: note = Synthetic oligonucleotide
  source 1..19: mol_type = other DNA
caggctggactgcggactg

SEQ ID NO: 7 - DNA, 20 bp, synthetic construct
  misc_feature 1..20: note = Synthetic oligonucleotide
  source 1..20: mol_type = other DNA
aatgcctgctccccttcacc

SEQ ID NO: 8 - DNA, 22 bp, synthetic construct
  misc_feature 1..22: note = Synthetic oligonucleotide
  source 1..22: mol_type = other DNA
ttgtctcatggttccacaagcc

SEQ ID NO: 9 - DNA, 21 bp, synthetic construct
  misc_feature 1..21: note = Synthetic oligonucleotide
  source 1..21: mol_type = other DNA
tgttaacaagagcctgggacc

SEQ ID NO: 10 - DNA, 20 bp, synthetic construct
  misc_feature 1..20: note = Synthetic oligonucleotide
  source 1..20: mol_type = other DNA
ggtgaaggggagcaggcatt

SEQ ID NO: 11 - DNA, 20 bp, synthetic construct
  misc_feature 1..20: note = Synthetic oligonucleotide
  source 1..20: mol_type = other DNA
tgtcatgagagctctgccct

SEQ ID NO: 12 - DNA, 22 bp, synthetic construct
  misc_feature 1..22: note = Synthetic oligonucleotide
  source 1..22: mol_type = other DNA
cgcctctcacgaaaactaaccc

SEQ ID NO: 13 - DNA, 20 bp, synthetic construct
  misc_feature 1..20: note = Synthetic oligonucleotide
  source 1..20: mol_type = other DNA
ggacctccctcactagcccc

SEQ ID NO: 14 - DNA, 22 bp, synthetic construct
  misc_feature 1..22: note = Synthetic oligonucleotide
  source 1..22: mol_type = other DNA
cactcatggcagaaggtgaagg

SEQ ID NO: 15 - DNA, 24 bp, synthetic construct
  misc_feature 1..24: note = Synthetic oligonucleotide
  source 1..24: mol_type = other DNA
tagatctccagcatcacatccctg

SEQ ID NO: 16 - DNA, 20 bp, synthetic construct
  misc_feature 1..20: note = Synthetic oligonucleotide
  source 1..20: mol_type = other DNA
caggaggagtggcagcaact

SEQ ID NO: 17 - DNA, 20 bp, synthetic construct
  misc_feature 1..20: note = Synthetic oligonucleotide
  source 1..20: mol_type = other DNA
cccggcagttcttccttcca

SEQ ID NO: 18 - DNA, 20 bp, synthetic construct
  misc_feature 1..20: note = Synthetic oligonucleotide
  source 1..20: mol_type = other DNA
tgaggacaaggggcacaagg

SEQ ID NO: 19 - DNA, 20 bp, synthetic construct
  misc_feature 1..20: note = Synthetic oligonucleotide
  source 1..20: mol_type = other DNA
gccccaacctgcctctaaca

SEQ ID NO: 20 - DNA, 20 bp, synthetic construct
  misc_feature 1..20: note = Synthetic oligonucleotide
  source 1..20: mol_type = other DNA
gtcctgcccagaagacagca

SEQ ID NO: 21 - DNA, 20 bp, synthetic construct
  misc_feature 1..20: note = Synthetic oligonucleotide
  source 1..20: mol_type = other DNA
ctgtggggatggggtggaag

SEQ ID NO: 22 - DNA, 20 bp, synthetic construct
  misc_feature 1..20: note = Synthetic oligonucleotide
  source 1..20: mol_type = other DNA
cccagtctcgtctcgcctac

SEQ ID NO: 23 - DNA, 20 bp, synthetic construct
  misc_feature 1..20: note = Synthetic oligonucleotide
  source 1..20: mol_type = other DNA
acccttctgccagcttgtga

SEQ ID NO: 24 - DNA, 20 bp, synthetic construct
  misc_feature 1..20: note = Synthetic oligonucleotide
  source 1..20: mol_type = other DNA
ggcccctatgtgcttgctca

SEQ ID NO: 25 - DNA, 20 bp, synthetic construct
  misc_feature 1..20: note = Synthetic oligonucleotide
  source 1..20: mol_type = other DNA
tgagcaagcacataggggcc

SEQ ID NO: 26 - DNA, 20 bp, synthetic construct
  misc_feature 1..20: note = Synthetic oligonucleotide
  source 1..20: mol_type = other DNA
gctcctgtctgccttttccg

SEQ ID NO: 27 - DNA, 20 bp, synthetic construct
  misc_feature 1..20: note = Synthetic oligonucleotide
  source 1..20: mol_type = other DNA
acacggattctggcagctgt

SEQ ID NO: 28 - DNA, 19 bp, synthetic construct
  misc_feature 1..19: note = Synthetic oligonucleotide
  source 1..19: mol_type = other DNA
gattgcagcaccccaaccc

SEQ ID NO: 29 - DNA, 20 bp, synthetic construct
  misc_feature 1..20: note = Synthetic oligonucleotide
  source 1..20: mol_type = other DNA
aagcttttgaggtgggcgtg

SEQ ID NO: 30 - DNA, 20 bp, synthetic construct
  misc_feature 1..20: note = Synthetic oligonucleotide
  source 1..20: mol_type = other DNA
atgcccatctcagcctacca

SEQ ID NO: 31 - DNA, 20 bp, synthetic construct
  misc_feature 1..20: note = Synthetic oligonucleotide
  source 1..20: mol_type = other DNA
gacatcatggggtggggagg

SEQ ID NO: 32 - DNA, 20 bp, synthetic construct
  misc_feature 1..20: note = Synthetic oligonucleotide
  source 1..20: mol_type = other DNA
tatcactgtcctcgcctgga

SEQ ID NO: 33 - DNA, 20 bp, synthetic construct
  misc_feature 1..20: note = Synthetic oligonucleotide
  source 1..20: mol_type = other DNA
tctgcaggtcctcatcagcg

SEQ ID NO: 34 - DNA, 20 bp, synthetic construct
  misc_feature 1..20: note = Synthetic oligonucleotide
  source 1..20: mol_type = other DNA
ccccactaccctgcactgag

SEQ ID NO: 35 - DNA, 20 bp, synthetic construct
  misc_feature 1..20: note = Synthetic oligonucleotide
  source 1..20: mol_type = other DNA
gtgtgttggcagtgggagga

SEQ ID NO: 36 - DNA, 20 bp, synthetic construct
  misc_feature 1..20: note = Synthetic oligonucleotide
  source 1..20: mol_type = other DNA
cctccattccaccgactccc

SEQ ID NO: 37 - DNA, 20 bp, synthetic construct
  misc_feature 1..20: note = Synthetic oligonucleotide
  source 1..20: mol_type = other DNA
gctcccttcccagatgctgt

SEQ ID NO: 38 - DNA, 20 bp, synthetic construct
  misc_feature 1..20: note = Synthetic oligonucleotide
  source 1..20: mol_type = other DNA
tggccagttgagctcaggaa

SEQ ID NO: 39 - DNA, 20 bp, synthetic construct
  misc_feature 1..20: note = Synthetic oligonucleotide
  source 1..20: mol_type = other DNA
tgtgcacggtggaaacctgt

SEQ ID NO: 40 - DNA, 20 bp, synthetic construct
  misc_feature 1..20: note = Synthetic oligonucleotide
  source 1..20: mol_type = other DNA
tcctgcaagccactacccaa

SEQ ID NO: 41 - DNA, 20 bp, synthetic construct
  misc_feature 1..20: note = Synthetic oligonucleotide
  source 1..20: mol_type = other DNA
tgttggcagtgggaggaaca

SEQ ID NO: 42 - DNA, 20 bp, synthetic construct
  misc_feature 1..20: note = Synthetic oligonucleotide
  source 1..20: mol_type = other DNA
cctcctccattccaccgact

SEQ ID NO: 43 - DNA, 21 bp, synthetic construct
  misc_feature 1..21: note = Synthetic oligonucleotide
  source 1..21: mol_type = other DNA
ggtctcgttcacctcctcacg

SEQ ID NO: 44 - DNA, 20 bp, synthetic construct
  misc_feature 1..20: note = Synthetic oligonucleotide
  source 1..20: mol_type = other DNA
cgctgatgaggacctgcaga

SEQ ID NO: 45 - DNA, 20 bp, synthetic construct
  misc_feature 1..20: note = Synthetic oligonucleotide
  source 1..20: mol_type = other DNA
gtgagcacgccattggaaca

SEQ ID NO: 46 - DNA, 20 bp, synthetic construct
  misc_feature 1..20: note = Synthetic oligonucleotide
  source 1..20: mol_type = other DNA
agcgaagtgggaaatggctg

SEQ ID NO: 47 - DNA, 19 bp, synthetic construct
  misc_feature 1..19: note = Synthetic oligonucleotide
  source 1..19: mol_type = other DNA
gggtgtgttggcagtggga

SEQ ID NO: 48 - DNA, 20 bp, synthetic construct
  misc_feature 1..20: note = Synthetic oligonucleotide
  source 1..20: mol_type = other DNA
cattccaccgactcccagca

SEQ ID NO: 49 - DNA, 20 bp, synthetic construct
  misc_feature 1..20: note = Synthetic oligonucleotide
  source 1..20: mol_type = other DNA
accagggaaaggggagcatc

SEQ ID NO: 50 - DNA, 20 bp, synthetic construct
  misc_feature 1..20: note = Synthetic oligonucleotide
  source 1..20: mol_type = other DNA
ccggggaaggaagggttagc

SEQ ID NO: 51 - DNA, 20 bp, synthetic construct
  misc_feature 1..20: note = Synthetic oligonucleotide
  source 1..20: mol_type = other DNA
gtggcaatctggtaggctgc

SEQ ID NO: 52 - DNA, 20 bp, synthetic construct
  misc_feature 1..20: note = Synthetic oligonucleotide
  source 1..20: mol_type = other DNA
cccctgtcagcgcttacact

SEQ ID NO: 53 - DNA, 20 bp, synthetic construct
  misc_feature 1..20: note = Synthetic oligonucleotide
  source 1..20: mol_type = other DNA
ctttcactttcctgccggca

SEQ ID NO: 54 - DNA, 20 bp, synthetic construct
  misc_feature 1..20: note = Synthetic oligonucleotide
  source 1..20: mol_type = other DNA
ggacagccacctacagcgaa

SEQ ID NO: 55 - DNA, 20 bp, synthetic construct
  misc_feature 1..20: note = Synthetic oligonucleotide
  source 1..20: mol_type = other DNA
gcagatttccagttgccgct

SEQ ID NO: 56 - DNA, 20 bp, synthetic construct
  misc_feature 1..20: note = Synthetic oligonucleotide
  source 1..20: mol_type = other DNA
aggccctcagactcacagtg

SEQ ID NO: 57 - DNA, 21 bp, synthetic construct
  misc_feature 1..21: note = Synthetic oligonucleotide
  source 1..21: mol_type = other DNA
tgagcacacctagccacttcc

SEQ ID NO: 58 - DNA, 20 bp, synthetic construct
  misc_feature 1..20: note = Synthetic oligonucleotide
  source 1..20: mol_type = other DNA
tcccacaccactatcctgcg

SEQ ID NO: 59 - DNA, 20 bp, synthetic construct
  misc_feature 1..20: note = Synthetic oligonucleotide
  source 1..20: mol_type = other DNA
agatgatcaacagggcccgc

SEQ ID NO: 60 - DNA, 20 bp, synthetic construct
  misc_feature 1..20: note = Synthetic oligonucleotide
  source 1..20: mol_type = other DNA
tcaggacccagatagcacgg

SEQ ID NO: 61 - DNA, 20 bp, synthetic construct
  misc_feature 1..20: note = Synthetic oligonucleotide
  source 1..20: mol_type = other DNA
acttccagggccaatgacgg

SEQ ID NO: 62 - DNA, 21 bp, synthetic construct
  misc_feature 1..21: note = Synthetic oligonucleotide
  source 1..21: mol_type = other DNA
ccaacatgtacctgtggctgc

SEQ ID NO: 63 - DNA, 20 bp, synthetic construct
  misc_feature 1..20: note = Synthetic oligonucleotide
  source 1..20: mol_type = other DNA
tcaagcagtgagaacagggc

SEQ ID NO: 64 - DNA, 20 bp, synthetic construct
  misc_feature 1..20: note = Synthetic oligonucleotide
  source 1..20: mol_type = other DNA
aaggatggggctgatggtgg

SEQ ID NO: 65 - DNA, 20 bp, synthetic construct
  misc_feature 1..20: note = Synthetic oligonucleotide
  source 1..20: mol_type = other DNA
tccacccacatccacaaccc

SEQ ID NO: 66 - DNA, 20 bp, synthetic construct
  misc_feature 1..20: note = Synthetic oligonucleotide
  source 1..20: mol_type = other DNA
tgcttctcaacagtggccga

SEQ ID NO: 67 - DNA, 20 bp, synthetic construct
  misc_feature 1..20: note = Synthetic oligonucleotide
  source 1..20: mol_type = other DNA
ctcaccctgaaccccttgca

SEQ ID NO: 68 - DNA, 20 bp, synthetic construct
  misc_feature 1..20: note = Synthetic oligonucleotide
  source 1..20: mol_type = other DNA
gcaggggtggaatggagtgt

SEQ ID NO: 69 - DNA, 20 bp, synthetic construct
  misc_feature 1..20: note = Synthetic oligonucleotide
  source 1..20: mol_type = other DNA
tgcccttcactggagagcag

SEQ ID NO: 70 - DNA, 20 bp, synthetic construct
  misc_feature 1..20: note = Synthetic oligonucleotide
  source 1..20: mol_type = other DNA
ctgctggggtttctggggaa

SEQ ID NO: 71 - DNA, 20 bp, synthetic construct
  misc_feature 1..20: note = Synthetic oligonucleotide
  source 1..20: mol_type = other DNA
ccactctcccagcctgtgac

SEQ ID NO: 72 - DNA, 20 bp, synthetic construct
  misc_feature 1..20: note = Synthetic oligonucleotide
  source 1..20: mol_type = other DNA
cgtgggtagctgtgtggaca

SEQ ID NO: 73 - DNA, 20 bp, synthetic construct
  misc_feature 1..20: note = Synthetic oligonucleotide
  source 1..20: mol_type = other DNA
tgtccacacagctacccacg

SEQ ID NO: 74 - DNA, 20 bp, synthetic construct
  misc_feature 1..20: note = Synthetic oligonucleotide
  source 1..20: mol_type = other DNA
ggcgtgtctcagtgggaaga

SEQ ID NO: 75 - DNA, 20 bp, synthetic construct
  misc_feature 1..20: note = Synthetic oligonucleotide
  source 1..20: mol_type = other DNA
ctgagtgtgtctgtgggcca

SEQ ID NO: 76 - DNA, 20 bp, synthetic construct
  misc_feature 1..20: note = Synthetic oligonucleotide
  source 1..20: mol_type = other DNA
aggacaagcagagacacgca

SEQ ID NO: 77 - DNA, 20 bp, synthetic construct
  misc_feature 1..20: note = Synthetic oligonucleotide
  source 1..20: mol_type = other DNA
gctatgttgggctggagggt

SEQ ID NO: 78 - DNA, 20 bp, synthetic construct
  misc_feature 1..20: note = Synthetic oligonucleotide
  source 1..20: mol_type = other DNA
gagcgccacatgtcctctga

SEQ ID NO: 79 - DNA, 20 bp, synthetic construct
  misc_feature 1..20: note = Synthetic oligonucleotide
  source 1..20: mol_type = other DNA
cctgggaccctgagttgcaa

SEQ ID NO: 80 - DNA, 20 bp, synthetic construct
  misc_feature 1..20: note = Synthetic oligonucleotide
  source 1..20: mol_type = other DNA
ccatgggcctgagtcactcc

SEQ ID NO: 81 - DNA, 20 bp, synthetic construct
  misc_feature 1..20: note = Synthetic oligonucleotide
  source 1..20: mol_type = other DNA
tttcctctctcccgccctct

SEQ ID NO: 82 - DNA, 20 bp, synthetic construct
  misc_feature 1..20: note = Synthetic oligonucleotide
  source 1..20: mol_type = other DNA
tgcgggagtttgggaccttt

SEQ ID NO: 83 - DNA, 20 bp, synthetic construct
  misc_feature 1..20: note = Synthetic oligonucleotide
  source 1..20: mol_type = other DNA
aggcttgcctgggaagtgaa

SEQ ID NO: 84 - DNA, 20 bp, synthetic construct
  misc_feature 1..20: note = Synthetic oligonucleotide
  source 1..20: mol_type = other DNA
cctggggtggtgctttgaga

SEQ ID NO: 85 - DNA, 20 bp, synthetic construct
  misc_feature 1..20: note = Synthetic oligonucleotide
  source 1..20: mol_type = other DNA
tccccgcccaaatctcatgt

SEQ ID NO: 86 - DNA, 20 bp, synthetic construct
  misc_feature 1..20: note = Synthetic oligonucleotide
  source 1..20: mol_type = other DNA
acccaaacaccctccaccag

SEQ ID NO: 87 - DNA, 23 bp, synthetic construct
  misc_feature 1..23: note = Synthetic oligonucleotide
  source 1..23: mol_type = other DNA
gaaagccctctctgtgcctgaat

SEQ ID NO: 88 - DNA, 21 bp, synthetic construct
  misc_feature 1..21: note = Synthetic oligonucleotide
  source 1..21: mol_type = other DNA
agaactgctctcggacaaaca

SEQ ID NO: 89 - DNA, 20 bp, synthetic construct
  misc_feature 1..20: note = Synthetic oligonucleotide
  source 1..20: mol_type = other DNA
aagcggcacagaaacatcgg

SEQ ID NO: 90 - DNA, 20 bp, synthetic construct
  misc_feature 1..20: note = Synthetic oligonucleotide
  source 1..20: mol_type = other DNA
cccagcccagcctacttctt

SEQ ID NO: 91 - DNA, 20 bp, synthetic construct
  misc_feature 1..20: note = Synthetic oligonucleotide
  source 1..20: mol_type = other DNA
cagctacgtgactcagggca

SEQ ID NO: 92 - DNA, 21 bp, synthetic construct
  misc_feature 1..21: note = Synthetic oligonucleotide
  source 1..21: mol_type = other DNA
tgcctcccatcttgccaatga

SEQ ID NO: 93 - DNA, 20 bp, synthetic construct
  misc_feature 1..20: note = Synthetic oligonucleotide
  source 1..20: mol_type = other DNA
cagaccgagggctccaacac

SEQ ID NO: 94 - DNA, 20 bp, synthetic construct
  misc_feature 1..20: note = Synthetic oligonucleotide
  source 1..20: mol_type = other DNA
tctctctgggaaaacgcggt

SEQ ID NO: 95 - DNA, 20 bp, synthetic construct
  misc_feature 1..20: note = Synthetic oligonucleotide
  source 1..20: mol_type = other DNA
gagcagagtgaaggggcagt

SEQ ID NO: 96 - DNA, 19 bp, synthetic construct
  misc_feature 1..19: note = Synthetic oligonucleotide
  source 1..19: mol_type = other DNA
cctggtggtctggtctgcc

SEQ ID NO: 97 - DNA, 20 bp, synthetic construct
  misc_feature 1..20: note = Synthetic oligonucleotide
  source 1..20: mol_type = other DNA
tcactgggcaaggaagaggc

SEQ ID NO: 98 - DNA, 20 bp, synthetic construct
  misc_feature 1..20: note = Synthetic oligonucleotide
  source 1..20: mol_type = other DNA
ccttctgtctcctgagcccc

SEQ ID NO: 99 - DNA, 20 bp, synthetic construct
  misc_feature 1..20: note = Synthetic oligonucleotide
  source 1..20: mol_type = other DNA
caagcaccgcgttttcccag

SEQ ID NO: 100 - DNA, 20 bp, synthetic construct
  misc_feature 1..20: note = Synthetic oligonucleotide
  source 1..20: mol_type = other DNA
cctggggtggtgctttgaga

SEQ ID NO: 101 - DNA, 20 bp, synthetic construct
  misc_feature 1..20: note = Synthetic oligonucleotide
  source 1..20: mol_type = other DNA
gcaaggaagaggccaagggt

SEQ ID NO: 102 - DNA, 20 bp, synthetic construct
  misc_feature 1..20: note = Synthetic oligonucleotide
  source 1..20: mol_type = other DNA
ggcccactgtcccttctgtc

SEQ ID NO: 103 - DNA, 20 bp, synthetic construct
  misc_feature 1..20: note = Synthetic oligonucleotide
  source 1..20: mol_type = other DNA
tcggaaacagccagaggagg

SEQ ID NO: 104 - DNA, 20 bp, synthetic construct
  misc_feature 1..20: note = Synthetic oligonucleotide
  source 1..20: mol_type = other DNA
actcggatggcagggtgaat

SEQ ID NO: 105 - DNA, 20 bp, synthetic construct
  misc_feature 1..20: note = Synthetic oligonucleotide
  source 1..20: mol_type = other DNA
aggcttgcctgggaagtgaa

SEQ ID NO: 106 - DNA, 21 bp, synthetic construct
  misc_feature 1..21: note = Synthetic oligonucleotide
  source 1..21: mol_type = other DNA
cgggttcttagcgtcctcact

SEQ ID NO: 107 - DNA, 21 bp, synthetic construct
  misc_feature 1..21: note = Synthetic oligonucleotide
  source 1..21: mol_type = other DNA
tgggaggtggcatttgagagg

SEQ ID NO: 108 - DNA, 20 bp, synthetic construct
  misc_feature 1..20: note = Synthetic oligonucleotide
  source 1..20: mol_type = other DNA
ccctctgccaccatttcagc

SEQ ID NO: 109 - DNA, 20 bp, synthetic construct
  misc_feature 1..20: note = Synthetic oligonucleotide
  source 1..20: mol_type = other DNA
agaagaagcaccgcacgagt

SEQ ID NO: 110 - DNA, 21 bp, synthetic construct
  misc_feature 1..21: note = Synthetic oligonucleotide
  source 1..21: mol_type = other DNA
gggacatttcagactggcacc

SEQ ID NO: 111 - DNA, 20 bp, synthetic construct
  misc_feature 1..20: note = Synthetic oligonucleotide
  source 1..20: mol_type = other DNA
tgcccaacccatttctccct

SEQ ID NO: 112 - DNA, 20 bp, synthetic construct
  misc_feature 1..20: note = Synthetic oligonucleotide
  source 1..20: mol_type = other DNA
tggattctgtgggcttgcag

SEQ ID NO: 113 - DNA, 20 bp, synthetic construct
  misc_feature 1..20: note = Synthetic oligonucleotide
  source 1..20: mol_type = other DNA
tccaccccactctgaggtca

SEQ ID NO: 114 - DNA, 20 bp, synthetic construct
  misc_feature 1..20: note = Synthetic oligonucleotide
  source 1..20: mol_type = other DNA
gaggtgggagtgggattcgg

SEQ ID NO: 115 - DNA, 26 bp, synthetic construct
  misc_feature 1..26: note = Synthetic oligonucleotide
  source 1..26: mol_type = other DNA
gtgaacctagcgtcatacagcctaag

SEQ ID NO: 116 - DNA, 24 bp, synthetic construct
  misc_feature 1..24: note = Synthetic oligonucleotide
  source 1..24: mol_type = other DNA
ttgacttgccttatctgatccccg

SEQ ID NO: 117 - DNA, 20 bp, synthetic construct
  misc_feature 1..20: note = Synthetic oligonucleotide
  source 1..20: mol_type = other DNA
aaacacacagggccgaggaa

SEQ ID NO: 118 - DNA, 20 bp, synthetic construct
  misc_feature 1..20: note = Synthetic oligonucleotide
  source 1..20: mol_type = other DNA
aagtgactggcctcctgagc

SEQ ID NO: 119 - DNA, 20 bp, synthetic construct
  misc_feature 1..20: note = Synthetic oligonucleotide
  source 1..20: mol_type = other DNA
ccacttgtccctaccccacc

SEQ ID NO: 120 - DNA, 20 bp, synthetic construct
  misc_feature 1..20: note = Synthetic oligonucleotide
  source 1..20: mol_type = other DNA
acttaacaggtcgcgctggt

SEQ ID NO: 121 - DNA, 20 bp, synthetic construct
  misc_feature 1..20: note = Synthetic oligonucleotide
  source 1..20: mol_type = other DNA
gcacaaccgcacacccatac

SEQ ID NO: 122 - DNA, 20 bp, synthetic construct
  misc_feature 1..20: note = Synthetic oligonucleotide
  source 1..20: mol_type = other DNA
cgtgggtagctgtgtggaca

SEQ ID NO: 123 - DNA, 20 bp, synthetic construct
  misc_feature 1..20: note = Synthetic oligonucleotide
  source 1..20: mol_type = other DNA
ctcctccacctgctgttccc

SEQ ID NO: 124 - DNA, 20 bp, synthetic construct
  misc_feature 1..20: note = Synthetic oligonucleotide
  source 1..20: mol_type = other DNA
tgcaggcgtgtaaatgtggc

SEQ ID NO: 125 - DNA, 20 bp, synthetic construct
  misc_feature 1..20: note = Synthetic oligonucleotide
  source 1..20: mol_type = other DNA
ctgcacgccactcaacactc

SEQ ID NO: 126 - DNA, 20 bp, synthetic construct
  misc_feature 1..20: note = Synthetic oligonucleotide
  source 1..20: mol_type = other DNA
gggaacagcaggtggaggag

SEQ ID NO: 127 - DNA, 20 bp, synthetic construct
  misc_feature 1..20: note = Synthetic oligonucleotide
  source 1..20: mol_type = other DNA
atggaggcttggctgtgagg

SEQ ID NO: 128 - DNA, 20 bp, synthetic construct
  misc_feature 1..20: note = Synthetic oligonucleotide
  source 1..20: mol_type = other DNA
agaagacgcagcgcttggta

SEQ ID NO: 129 - DNA, 21 bp, synthetic construct
  misc_feature 1..21: note = Synthetic oligonucleotide
  source 1..21: mol_type = other DNA
agctctgccctcatgaatgct

SEQ ID NO: 130 - DNA, 22 bp, synthetic construct
  misc_feature 1..22: note = Synthetic oligonucleotide
  source 1..22: mol_type = other DNA
accctcctgtactcctttgtcc

SEQ ID NO: 131 - DNA, 22 bp, synthetic construct
  misc_feature 1..22: note = Synthetic oligonucleotide
  source 1..22: mol_type = other DNA
agaggggtgatagctaaagggt

SEQ ID NO: 132 - DNA, 20 bp, synthetic construct
  misc_feature 1..20: note = Synthetic oligonucleotide
  source 1..20: mol_type = other DNA
ggtgggttgaggggctagtg

SEQ ID NO: 133 - DNA, 21 bp, synthetic construct
  misc_feature 1..21: note = Synthetic oligonucleotide
  source 1..21: mol_type = other DNA
tcttgtctcccatctgccaca

SEQ ID NO: 134 - DNA, 22 bp, synthetic construct
  misc_feature 1..22: note = Synthetic oligonucleotide
  source 1..22: mol_type = other DNA
tgtacagggatgtgatgctgga

SEQ ID NO: 135 - DNA, 20 bp, synthetic construct
  misc_feature 1..20: note = Synthetic oligonucleotide
  source 1..20: mol_type = other DNA
gatggcaatctgggtggaga

SEQ ID NO: 136 - DNA, 22 bp, synthetic construct
  misc_feature 1..22: note = Synthetic oligonucleotide
  source 1..22: mol_type = other DNA
ttgtccaatccagtgcctctcc

SEQ ID NO: 137 - DNA, 24 bp, synthetic construct
  misc_feature 1..24: note = Synthetic oligonucleotide
  source 1..24: mol_type = other DNA
gggtgatagctaaagggtacaggg

SEQ ID NO: 138 - DNA, 20 bp, synthetic construct
  misc_feature 1..20: note = Synthetic oligonucleotide
  source 1..20: mol_type = other DNA
atatggtgggttgaggggct

SEQ ID NO: 139 - DNA, 21 bp, synthetic construct
  misc_feature 1..21: note = Synthetic oligonucleotide
  source 1..21: mol_type = other DNA
aggtgggacttagtgggaggt

SEQ ID NO: 140 - DNA, 24 bp, synthetic construct
  misc_feature 1..24: note = Synthetic oligonucleotide
  source 1..24: mol_type = other DNA
tcgctaggcttacttgacttcagt

SEQ ID NO: 141 - DNA, 22 bp, synthetic construct
  misc_feature 1..22: note = Synthetic oligonucleotide
  source 1..22: mol_type = other DNA
ccatggctcttctccttgttcc

SEQ ID NO: 142 - DNA, 20 bp, synthetic construct
  misc_feature 1..20: note = Synthetic oligonucleotide
  source 1..20: mol_type = other DNA
tttgaggatgtggctgtgga

SEQ ID NO: 143 - DNA, 24 bp, synthetic construct
  misc_feature 1..24: note = Synthetic oligonucleotide
  source 1..24: mol_type = other DNA
gctaaataatgccagtctgccagc

SEQ ID NO: 144 - DNA, 21 bp, synthetic construct
  misc_feature 1..21: note = Synthetic oligonucleotide
  source 1..21: mol_type = other DNA
tcttgaatggcagcagtgtcg

SEQ ID NO: 145 - DNA, 20 bp, synthetic construct
  misc_feature 1..20: note = Synthetic oligonucleotide
  source 1..20: mol_type = other DNA
aaccgtggcattggaaggga

SEQ ID NO: 146 - DNA, 20 bp, synthetic construct
  misc_feature 1..20: note = Synthetic oligonucleotide
  source 1..20: mol_type = other DNA
gggtgtgactgagagggagc

SEQ ID NO: 147 - DNA, 20 bp, synthetic construct
  misc_feature 1..20: note = Synthetic oligonucleotide
  source 1..20: mol_type = other DNA
tgggagtaggcgagacgaga

SEQ ID NO: 148 - DNA, 20 bp, synthetic construct
  misc_feature 1..20: note = Synthetic oligonucleotide
  source 1..20: mol_type = other DNA
tggcagaagggtgggctttt

SEQ ID NO: 149 - DNA, 20 bp, synthetic construct
  misc_feature 1..20: note = Synthetic oligonucleotide
  source 1..20: mol_type = other DNA
cacacacacacacacccacg

SEQ ID NO: 150 - DNA, 20 bp, synthetic construct
  misc_feature 1..20: note = Synthetic oligonucleotide
  source 1..20: mol_type = other DNA
tgtccgcagagcacaagagg

SEQ ID NO: 151 - DNA, 20 bp, synthetic construct
  misc_feature 1..20: note = Synthetic oligonucleotide
  source 1..20: mol_type = other DNA
gtaggcgagacgagactggg

SEQ ID NO: 152 - DNA, 20 bp, synthetic construct
  misc_feature 1..20: note = Synthetic oligonucleotide
  source 1..20: mol_type = other DNA
tcacaagctggcagaagggt

SEQ ID NO: 153 - DNA, 20 bp, synthetic construct
  misc_feature 1..20: note = Synthetic oligonucleotide
  source 1..20: mol_type = other DNA
ttgagattggcgtggggtca

SEQ ID NO: 154 - DNA, 20 bp, synthetic construct
  misc_feature 1..20: note = Synthetic oligonucleotide
  source 1..20: mol_type = other DNA
cactccagaggcacacaggg

SEQ ID NO: 155 - DNA, 20 bp, synthetic construct
  misc_feature 1..20: note = Synthetic oligonucleotide
  source 1..20: mol_type = other DNA
ttggggcagtggtaggtgag

SEQ ID NO: 156 - DNA, 20 bp, synthetic construct
  misc_feature 1..20: note = Synthetic oligonucleotide
  source 1..20: mol_type = other DNA
atcggattgcagcaccccaa

SEQ ID NO: 157 - DNA, 20 bp, synthetic construct
  misc_feature 1..20: note = Synthetic oligonucleotide
  source 1..20: mol_type = other DNA
tgcccagattcccaccttca

SEQ ID NO: 158 - DNA, 20 bp, synthetic construct
  misc_feature 1..20: note = Synthetic oligonucleotide
  source 1..20: mol_type = other DNA
ctcacctcccagcttccacc

SEQ ID NO: 159 - DNA, 20 bp, synthetic construct
  misc_feature 1..20: note = Synthetic oligonucleotide
  source 1..20: mol_type = other DNA
gctgtcatggagcgctgtct

SEQ ID NO: 160 - DNA, 20 bp, synthetic construct
  misc_feature 1..20: note = Synthetic oligonucleotide
  source 1..20: mol_type = other DNA
cccagtctcgtctcgcctac

SEQ ID NO: 161 - DNA, 20 bp, synthetic construct
  misc_feature 1..20: note = Synthetic oligonucleotide
  source 1..20: mol_type = other DNA
ctggctctggatggcttgga

SEQ ID NO: 162 - DNA, 20 bp, synthetic construct
  misc_feature 1..20: note = Synthetic oligonucleotide
  source 1..20: mol_type = other DNA
caccctgtgttccaatggcg

SEQ ID NO: 163 - DNA, 20 bp, synthetic construct
  misc_feature 1..20: note = Synthetic oligonucleotide
  source 1..20: mol_type = other DNA
gggctgatgtggctagggat

SEQ ID NO: 164 - DNA, 20 bp, synthetic construct
  misc_feature 1..20: note = Synthetic oligonucleotide
  source 1..20: mol_type = other DNA
cctccattccaccgactccc

SEQ ID NO: 165 - DNA, 20 bp, synthetic construct
  misc_feature 1..20: note = Synthetic oligonucleotide
  source 1..20: mol_type = other DNA
ggatctggctctggatggct

SEQ ID NO: 166 - DNA, 20 bp, synthetic construct
  misc_feature 1..20: note = Synthetic oligonucleotide
  source 1..20: mol_type = other DNA
cctgtgttccaatggcgtgc

SEQ ID NO: 167 - DNA, 20 bp, synthetic construct
  misc_feature 1..20: note = Synthetic oligonucleotide
  source 1..20: mol_type = other DNA
gtgagcacgccattggaaca

SEQ ID NO: 168 - DNA, 20 bp, synthetic construct
  misc_feature 1..20: note = Synthetic oligonucleotide
  source 1..20: mol_type = other DNA
tcctgcaagccactacccaa

SEQ ID NO: 169 - DNA, 20 bp, synthetic construct
  misc_feature 1..20: note = Synthetic oligonucleotide
  source 1..20: mol_type = other DNA
acgtcgtggcttcaaagtgc

SEQ ID NO: 170 - DNA, 20 bp, synthetic construct
  misc_feature 1..20: note = Synthetic oligonucleotide
  source 1..20: mol_type = other DNA
agcgaagtgggaaatggctg

SEQ ID NO: 171 - DNA, 20 bp, synthetic construct
  misc_feature 1..20: note = Synthetic oligonucleotide
  source 1..20: mol_type = other DNA
catctcgggccatgacgtcc

SEQ ID NO: 172 - DNA, 20 bp, synthetic construct
  misc_feature 1..20: note = Synthetic oligonucleotide
  source 1..20: mol_type = other DNA
tctggagctggatgactggc

SEQ ID NO: 173 - DNA, 20 bp, synthetic construct
  misc_feature 1..20: note = Synthetic oligonucleotide
  source 1..20: mol_type = other DNA
ggtcgggctggtctcaaact

SEQ ID NO: 174 - DNA, 20 bp, synthetic construct
  misc_feature 1..20: note = Synthetic oligonucleotide
  source 1..20: mol_type = other DNA
gcaatatgaggctgggcaca

SEQ ID NO: 175 - DNA, 19 bp, synthetic construct
  misc_feature 1..19: note = Synthetic oligonucleotide
  source 1..19: mol_type = other DNA
gcccggtgtccatgtcctt

SEQ ID NO: 176 - DNA, 20 bp, synthetic construct
  misc_feature 1..20: note = Synthetic oligonucleotide
  source 1..20: mol_type = other DNA
tcctcccactgccaacacac

SEQ ID NO: 177 - DNA, 20 bp, synthetic construct
  misc_feature 1..20: note = Synthetic oligonucleotide
  source 1..20: mol_type = other DNA
gatccacccacctcagcctc

SEQ ID NO: 178 - DNA, 20 bp, synthetic construct
  misc_feature 1..20: note = Synthetic oligonucleotide
  source 1..20: mol_type = other DNA
ccgtcattggccctggaagt

SEQ ID NO: 179 - DNA, 20 bp, synthetic construct
  misc_feature 1..20: note = Synthetic oligonucleotide
  source 1..20: mol_type = other DNA
acttccagggccaatgacgg

SEQ ID NO: 180 - DNA, 20 bp, synthetic construct
  misc_feature 1..20: note = Synthetic oligonucleotide
  source 1..20: mol_type = other DNA
aattccccaagccagccact

SEQ ID NO: 181 - DNA, 20 bp, synthetic construct
  misc_feature 1..20: note = Synthetic oligonucleotide
  source 1..20: mol_type = other DNA
ccacctcagcctcccaaagt

SEQ ID NO: 182 - DNA, 20 bp, synthetic construct
  misc_feature 1..20: note = Synthetic oligonucleotide
  source 1..20: mol_type = other DNA
agtcctacccgtcattggcc

SEQ ID NO: 183 - DNA, 21 bp, synthetic construct
  misc_feature 1..21: note = Synthetic oligonucleotide
  source 1..21: mol_type = other DNA
aggttgtcgatgaactgggca

SEQ ID NO: 184 - DNA, 20 bp, synthetic construct
  misc_feature 1..20: note = Synthetic oligonucleotide
  source 1..20: mol_type = other DNA
cacacagccacacagccatc

SEQ ID NO: 185 - DNA, 20 bp, synthetic construct
  misc_feature 1..20: note = Synthetic oligonucleotide
  source 1..20: mol_type = other DNA
ccgcacacatgtcagggaga

SEQ ID NO: 186 - DNA, 21 bp, synthetic construct
  misc_feature 1..21: note = Synthetic oligonucleotide
  source 1..21: mol_type = other DNA
acaacttggaggctcaggacc

SEQ ID NO: 187 - DNA, 20 bp, synthetic construct
  misc_feature 1..20: note = Synthetic oligonucleotide
  source 1..20: mol_type = other DNA
ggtgagggtgggatgtgtca

SEQ ID NO: 188 - DNA, 20 bp, synthetic construct
  misc_feature 1..20: note = Synthetic oligonucleotide
  source 1..20: mol_type = other DNA
ttagcctgacgtgccatgag

SEQ ID NO: 189 - DNA, 19 bp, synthetic construct
  misc_feature 1..19: note = Synthetic oligonucleotide
  source 1..19: mol_type = other DNA
acccacctcagcctcccaa

SEQ ID NO: 190 - DNA, 20 bp, synthetic construct
  misc_feature 1..20: note = Synthetic oligonucleotide
  source 1..20: mol_type = other DNA
ctacccgtcattggccctgg

SEQ ID NO: 191 - DNA, 20 bp, synthetic construct
  misc_feature 1..20: note = Synthetic oligonucleotide
  source 1..20: mol_type = other DNA
ctggcccactacctcccaag

SEQ ID NO: 192 - DNA, 18 bp, synthetic construct
  misc_feature 1..18: note = Synthetic oligonucleotide
  source 1..18: mol_type = other DNA
cctgctcaacgtggccct

(19) United States
(12) Patent Application Publication
(10) Pub. No.: US 2025/0154577 A1
(43) Pub. Date: May 15, 2025

(54) COMPOSITIONS, KITS, AND METHODS FOR SHORT TANDEM REPEAT ANALYSIS

(71) Applicant: LIFE TECHNOLOGIES CORPORATION, Carlsbad, CA (US)

(72) Inventor: Robert E. Lagace, Oakland, CA (US)

(21) Appl. No.: 18/718,196

(22) PCT Filed: Dec. 14, 2022
(86) PCT No.: PCT/US2022/081552

§ 371 Date: Jun. 10, 2024

Related U.S. Application Data
(60) Provisional application No. 63/428,978, filed on Nov. 30, 2022.
(60) Provisional application No. 63/291,271, filed on Dec. 17, 2021.
(60) Provisional application No. 63/291,245, filed on Dec. 17, 2021.

(51) Int. Cl. C12Q 1/686; C12Q 1/6851; C12Q 1/6858; C12Q 1/6888

(52) U.S. Cl. CPC C12Q 1/686; C12Q 1/6851; C12Q 1/6858; C12Q 1/6888; C12Q 2600/16; C12Q 2600/166

(57) ABSTRACT

Disclosed are compositions, kits, and methods for use in applications involving electrophoretic separation of nucleic acids, including capillary electrophoresis applications in which nucleic acid samples are labelled with dyes, size-separated, and subjected to short tandem repeat (STR) analysis. A sample containing DNA is mixed with a composition that includes at least one set of primers that target an STR region of the sample nucleic acid, a set of short quantification primers (QS primers) that target a first multi-copy sequence (QS sequence) of the sample nucleic acid, and a set of long quantification primers (QL primers) that target a second multi-copy sequence (QL sequence) of the sample nucleic acid, wherein the QS sequence is shorter than the QL sequence.

This application claims the benefit of and priority to U.S. Provisional Patent Application No. 63/291,245 filed Dec. 17, 2021; 63/291,271 filed Dec. 17, 2021; and 63/428,978 filed Nov. 30, 2022. Each of the foregoing applications is incorporated herein by reference in its entirety.

### BACKGROUND

This disclosure relates to compositions, kits, and methods for use in applications involving electrophoretic separation of nucleic acids, including capillary electrophoresis applications in which nucleic acid samples are labelled with dyes, size-separated, and subjected to short tandem repeat (STR) analysis.

Rapid DNA testing is typically understood to mean a process that generates a DNA profile from a sample in a matter of hours rather than days and that requires limited human intervention. For example, law enforcement can obtain a cheek swab of an individual under arrest and insert the sample into a rapid DNA testing instrument. At present, such instruments provide a DNA profile within about two hours. The DNA profile may then be compared to DNA profile databases and/or to DNA samples associated with unsolved crimes. Rapid DNA testing is also utilized in forensic analysis, identification of human remains, crime scene investigations, and the identification or exclusion of suspects at the booking station.

Rapid DNA instruments typically utilize electrophoresis to separate DNA within a sample based on size, and to provide a DNA profile based on STR typing. STR loci are targeted with specified primers and are then amplified and labelled with dyes. Often, multiple dyes are used, each dye being specific to a particular locus or set of loci that are expected to sufficiently spread out once size separated. This allows for allele typing of multiple loci spread among the different dye channels. With enough loci analyzed, the pattern of alleles provides highly accurate identification of an individual. In the U.S., for example, STR typing procedures commonly analyze the standard core loci of the Combined DNA Index System (CODIS), referred to as the CODIS 20 (or the core loci of the previous CODIS 13 standard).

Rapid DNA applications are beneficial due to the relative speed in which they provide results. Results can be obtained while suspects are still in custody, reducing the risk that the suspect will flee and be unreachable after DNA results are obtained. Rapid DNA applications also reduce the number of processing steps, the number of people handling the sample, and the number of location transfers required to obtain results. This lowers the risk of sample contamination or mishandling.

However, rapid DNA applications also have several limitations. DNA samples obtained from crime scenes, for example, often contain environmental debris and may include mixed DNA from multiple individuals. Moreover, because the amount of collected DNA varies depending on the amount available at the scene, the method of obtaining the sample, the type of sample (e.g., blood vs. touch/trace DNA), and the skill of the person(s) collecting the sample, the results can often be noisy compared to a full laboratory analysis.

Further, variation in DNA profile peak heights resulting from the above factors and/or from the random variation associated with amplification of the DNA (i.e., “stochastic effects”) can hamper result quality. Peak height thresholds aim to manage the noise in the profile by only considering peaks above the threshold as representative of alleles. Higher thresholds can therefore eliminate more artifacts resulting from stochastic effects. However, DNA peaks below the threshold often represent real alleles, so a higher threshold also lowers the sensitivity of the analysis.

Ambiguities in the DNA profile resulting from such stochastic effects are exacerbated when the amount of DNA in the sample is low. However, conventional rapid DNA applications do not provide a means for quantitating the amount of DNA in the sample. Thus, a user may not know whether the level of DNA in a given sample is too low, or at what point it should be considered as too low, without additional testing and/or consultation with an expert.

In addition, rapid DNA testing may involve degraded DNA and/or inhibited amplification of DNA. The resulting DNA profiles may have low quality and/or may require interpretation by an expert to resolve issues and ambiguities. A typical booking station is unlikely to have such experts on hand and readily available. Where expert interpretation is required, the application becomes much less “rapid”, and the potential benefits of rapid testing are not realized.

Accordingly, there is an ongoing need for improvements in rapid DNA testing applications. The present application is directed to compositions, kits, and methods that represent, in at least some implementations, an improvement over conventional rapid DNA testing.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A illustrates an example capillary electrophoresis instrument as an example of one application in which the present compositions, kits, and methods may be utilized;

FIG. 1B illustrates an example STR electropherogram;

FIGS. 2A and 2B list example QS sequences and corresponding QS primers and example QL sequences and corresponding QL primers, respectively;

FIG. 3A illustrates an example STR electropherogram that includes a short quantification standard (QS standard), a long quantification standard (QL standard), a short internal quality control (IQCS), and a long internal quality control (IQCL);

FIG. 3B illustrates an example STR electropherogram resulting from use of the QS standard, QL standard, IQCS, and IQCL, the results indicating that the level of genomic DNA in the sample is relatively low;

FIG. 3C illustrates an example STR electropherogram resulting from use of the QS standard, QL standard, IQCS, and IQCL, the results indicating negative detection of genomic DNA in the sample;

FIG. 3D illustrates an example STR electropherogram resulting from use of the QS standard, QL standard, IQCS, and IQCL, the results indicating that genomic DNA in the sample is degraded;

FIG. 3E illustrates an example STR electropherogram resulting from use of the QS standard, QL standard, IQCS, and IQCL, the results indicating inhibited amplification of genomic DNA in the sample;

FIG. 4 is a multi-channel electropherogram showing results using QS and QL markers selected from those shown in FIGS. 2A and 2B;

FIG. 5A is a chart illustrating peak heights of STR profiles resulting from a blood dilution series that varied the volume of blood utilized in the rapid DNA analysis, the results showing that suitable peak heights were obtained at blood volumes as low as about 0.4 μL;

FIG. 5B is a chart illustrating peak height ratios for different blood volumes at various tested loci, the results showing suitable performance at blood volumes as low as about 0.4 μL;

FIG. 6A is a table comparing results of a conventional rapid DNA testing process to an improved process according to the present disclosure, the improved process being modified to use a different collection swab, a lower lysate volume, and lower number of PCR cycles, showing that the modified process reduced the number of flags while also providing effective peak heights;

FIGS. 6B and 6C are charts illustrating peak heights (FIG. 6B) and peak height ratios (FIG. 6C) at various loci, comparing a conventional rapid DNA testing process (“RI Cotton 1 μL blood”) to an improved process according to the present disclosure (“BH1 HydraFlock 1 μL blood”), the results showing that the improved process led to less variance in peak heights;

FIG. 7 illustrates results of DNA input level testing, showing changes in the QS standard and QL standard in the corresponding STR profiles at different levels of input DNA;

FIGS. 8A and 8B illustrate average peak heights for QS and QL sequences across different levels of gDNA input based on control DNA and liquid blood; and

FIG. 9 illustrates results of DNA degradation testing, showing greater changes to the QL standard than the QS standard in the corresponding STR profiles at progressively higher levels of DNA degradation.

### DETAILED DESCRIPTION

### Example Analysis Instrument

The present disclosure includes specific examples directed to rapid DNA analysis systems using capillary electrophoresis. However, the skilled person will understand, in light of this disclosure, that the compositions, kits, and methods described herein may be additionally or alternatively utilized in other applications such as traditional gel electrophoresis applications. In addition, although several examples are described in the context of rapid DNA analysis, it will be understood that the embodiments described herein may also be beneficially utilized in other nucleic acid analysis applications, including conventional laboratory analysis applications.

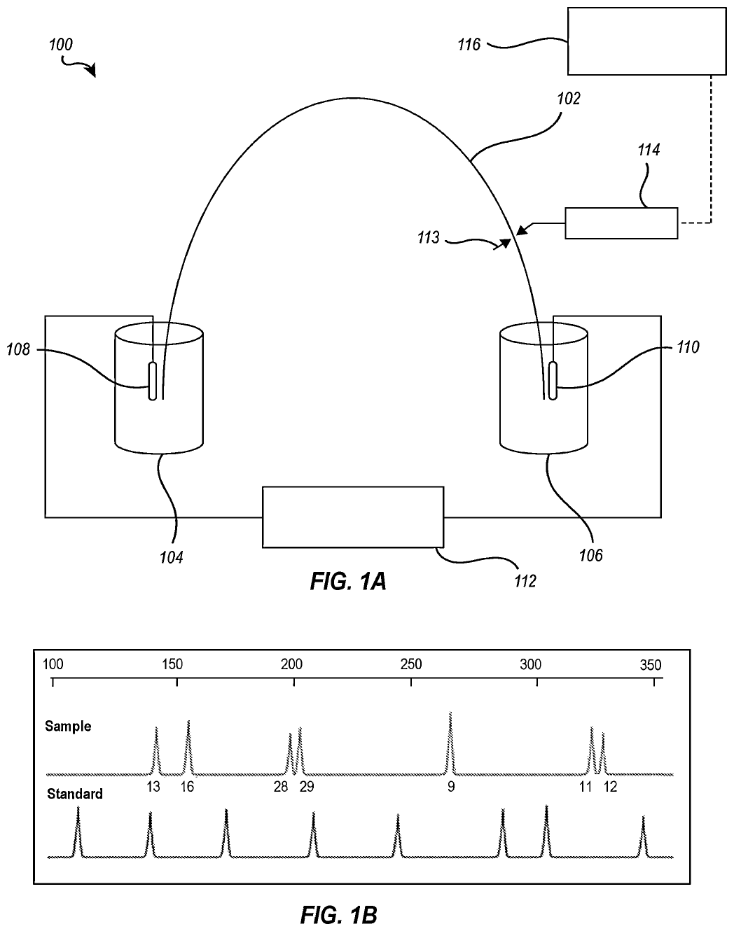

FIG. 1A schematically illustrates a capillary electrophoresis instrument 100 as one example of an application in which the presently described embodiments may be implemented. The capillary electrophoresis instrument 100 includes a capillary 102 with one end in contact with a source container 104 and the opposite end in contact with a destination container 106. The capillary 102 is filled with a sieving polymer. Typically, multiple capillaries are included in a capillary electrophoresis system to allow for parallel analysis of multiple samples, though for the sake of clarity only one capillary 102 is illustrated here. The use of capillaries, as opposed to conventional slab gels, allows for the use of stronger electrical fields, which results in faster separation and increased overall throughput.

The source container 104 and destination container 106 hold an appropriate electrolytic buffer, and the sample to be analyzed is added to the source container 104 or otherwise mixed with the buffer at the source container 104 when introduced into the capillary 102.

The capillary electrophoresis instrument 100 also includes a pair of electrodes 108 and 110 that are in electrical communication with the buffer solution at the opposing containers 104 and 106. A power supply 112 generates a voltage between the electrodes 108 and 110. The sample is introduced into the capillary 102 and the electric potential between electrodes 108 and 110 then causes the targeted analytes to migrate through the capillary 102 toward the destination container 106.

The analytes (e.g., nucleic acids) separate by size as they migrate through the capillary 102 according to differences in electrophoretic mobility through the capillary. That is, smaller fragments will move through the polymer faster than larger fragments. In applications where nucleic acids are the analytical target (as in this example), the negatively charged nucleic acid fragments will move from the negatively charged cathode 108 toward the positively charged anode 110 under the applied voltage.

The capillary 102 includes a detection window 113 coincident with a corresponding detector assembly 114. The detector assembly includes a laser and a fluorescence detector. As dye-labelled nucleic acid fragments pass through the detection window, the laser excites the dye-labelled fragments and the detector (e.g., a charge-coupled device (CCD) camera) detects the resulting fluorescence signals. Typically, multiple different dyes that each provide a different, known fluorescence response to the excitation light are used to label different nucleic acid targets. Thus, the identities of the fragments may be determined according to the character of the corresponding fluorescence signal.

The detector assembly 114 is communicatively coupled to a computer device 116. The computer device 116 includes one or more processors and memory (e.g., one or more hardware storage devices) that enable it to receive the fluorescence signal data from the detector assembly 114 and to generate an electropherogram showing the detected fluorescence signals of the different dye “channels” over time. Peaks in the electropherogram indicate times at which a labelled fragment passed through the detection window 113. By comparing these detected peaks to standard peaks (i.e., a “ladder”) from a sample having fragments of known size, the sizes of the fragments can be determined.

One major use of capillary electrophoresis is STR typing. STR loci are targeted with specified primers and are then amplified and labelled with dyes. Often, multiple dyes are used, each dye being specific to a particular locus or set of loci that are expected to sufficiently spread out once size separated. This allows for allele typing of multiple loci spread among the different dye channels. With enough loci analyzed, the pattern of alleles provides highly accurate identification of an individual. In the U.S., for example, STR typing procedures commonly analyze the standard core loci of the Combined DNA Index System (CODIS), referred to as the CODIS 20 (or the core loci of the previous CODIS 13 standard).

One example of a rapid DNA instrument that may be utilized with the disclosed compositions, kits, and methods is the RapidHIT ID System (Applied Biosystems, Catalog No. A41810). The RapidHIT ID System utilizes RapidINTEL™ sample cartridges (Applied Biosystems, Catalog No. A43942). The embodiments described herein may utilize similar components, dyes, STR target loci, and the like, but with the modifications described herein.

FIG. 1B is an exemplary STR electropherogram. Common components of such an electropherogram include a size standard showing peaks from nucleic acid fragments of known size, the peaks of the test sample in one or more dye channels (a single channel shown here), and a size indicator showing the corresponding length (in number of base pairs) of the fragments. As shown, the peaks of the test sample are usually also annotated with a number to indicate the STR allele(s) detected at the particular locus associated with each peak or pair of peaks. Different alleles for a given STR locus have different sizes due to different numbers of the repeat sequence present. Loci labels are not shown here but are often also included.

### Quantification Standards

Certain embodiments described herein utilize quantification standards to improve nucleic acid analysis, including STR analysis included as part of a rapid DNA testing process. The QS and QL standards are designed to target regions of the genomic DNA of the sample, and do not include synthetic and/or additionally added target sequences. The QS standard includes a set of short quantification primers (QS primers) that target a first sequence (QS sequence) of the sample nucleic acid, whereas the QL standard includes a set of long quantification primers (QL primers) that target a second sequence (QL sequence) of the sample nucleic acid. The QS sequence is shorter than the QL sequence.

The QS and QL primers are used with at least one set of primers that target an STR locus of the sample nucleic acid. In some embodiments, a composition includes the QS and QL primers and multiple sets of primers each configured to target a different STR region of the sample nucleic acid. Examples of suitable STR regions are known in the art, including those targeted with the GlobalFiler™ IQC PCR amplification kit (Applied Biosystems, Catalog No. A43565) and/or other STR loci commonly analyzed in the forensics, criminal investigation, and human identification fields.

In some embodiments, the QS sequence is shorter than the targeted STR regions and the QL sequence is longer than the targeted STR regions such that the QS standard and the QL standard bracket the targeted STR regions along the spectrum of analyzed sizes. However, in other embodiments, the QS and/or the QL sequence(s) have sizes that fall within the size range of the STR regions. In such embodiments, the QS and/or QL marker(s) preferably have their own unique size range and/or unique dye label to be sufficiently distinguished from the targeted, STR loci on the electropherogram.

The QS and/or QL sequences may be sequences that have a single copy within the human genome. In other embodiments, the QS and/or QL sequences are multi-copy sequences within the human genome. The QS and QL sequences are also preferably free of indels to prevent size variation across samples and/or across different regions of the multi-copy sequence.

In some embodiments, the QS sequence has a size ranging from about 40 to about 120 nucleotides, or about 50 to about 110 nucleotides, or about 60 to about 100 nucleotides, or about 70 to about 90 nucleotides, or a size within a range having endpoints defined by any two of the foregoing values. In some embodiments, the QL sequence has a size ranging from about 300 to about 600 nucleotides, or about 325 to about 575 nucleotides, or about 350 to about 550 nucleotides, or about 375 to about 525 nucleotides, or about 400 to about 500 nucleotides, or about 420 to about 480 nucleotides, or about 440 to about 460 nucleotides, or a size within a range having endpoints defined by any two of the foregoing values.

The sizes of the QS and QL sequences may be varied according to particular application needs, and need not fall within the ranges disclosed above. For example, depending on the particular STR regions targeted and the range of potential allele sizes for such targeted STR loci, the QS and QL sizes can be adjusted so as to bracket the targeted STR loci. Moreover, as discussed above, in certain embodiments the QS and/or QL sequence(s) have sizes that fall within the size range of the targeted STR regions, and the QS and QL sequences need not necessarily completely bracket all targeted STR loci.

In some embodiments, the set of QS primers includes a forward primer and a reverse primer to target the corresponding QS sequence. At least some of the forward primer molecules and/or at least some of the reverse primer molecules of the set of QS primers include a detectable label. In some embodiments, not all of the forward primer molecules and/or reverse primer molecules are labelled. Limiting the number of labelled molecules beneficially provides more control over the expected peak height of the QS standard.

For example, the ratio of labelled to non-labelled primers can be controlled to provide an effective peak height (e.g., not excessively short or excessively tall) while still providing sufficient overall levels of primers to drive effective amplification of the QS sequence. In some embodiments, the ratio of labelled to non-labelled forward primers in the set of QS primers is about 1:1 to about 1:5, such as about 1:1, 1:2, 1:2, 1:4, 1:5, or a range of ratios with any of the foregoing as endpoints, and/or the ratio of labelled to non-labelled reverse primers in the set of QS primers is about 1:1 to about 1:5, such as about 1:1, 1:2, 1:2, 1:4, 1:5, or a range of ratios with any of the foregoing as endpoints.

In some embodiments, the set of QL primers includes a forward primer and a reverse primer to target the corresponding QL sequence. At least some of the forward primer molecules and/or at least some of the reverse primer molecules of the set of QL primers include a detectable label. In some embodiments, not all of the forward primer molecules and/or reverse primer molecules are labelled. Limiting the number of labelled molecules beneficially provides more control over the expected peak height of the QL standard.

For example, the ratio of labelled to non-labelled primers can be controlled to provide an effective peak height (e.g., not excessively short or excessively tall) while still providing sufficient overall levels of primers to drive effective amplification of the QL sequence. In some embodiments, the ratio of labelled to non-labelled forward primers in the set of QL primers is about 1:1 to about 1:5, such as about 1:1, 1:2, 1:2, 1:4, 1:5, or a range with any of the foregoing ratios as endpoints, and/or the ratio of labelled to non-labelled reverse primers in the set of QL primers is about 1:1 to about 1:5, such as about 1:1, 1:2, 1:2, 1:4, 1:5, or a range with any of the foregoing ratios as endpoints.

The QS primers and/or QL primers described herein need not have 100% homology to their respective QS sequence and QL sequence targets to be effective, though in some embodiments, homology is substantially 100%. In some embodiments, one or more of the disclosed primers have a homology to their respective target of about 70%, about 80%, about 85%, about 90%, about 95%, about 97%, about 98%, about 99%, about 99.5%, up to substantially 100%, or a range with endpoints defined by any two of the foregoing percentage values. Some combinations of primers may include primers each with different homologies to their respective targets, and the homologies may be, for example, within a range with endpoints defined by any two of the foregoing percentage values.

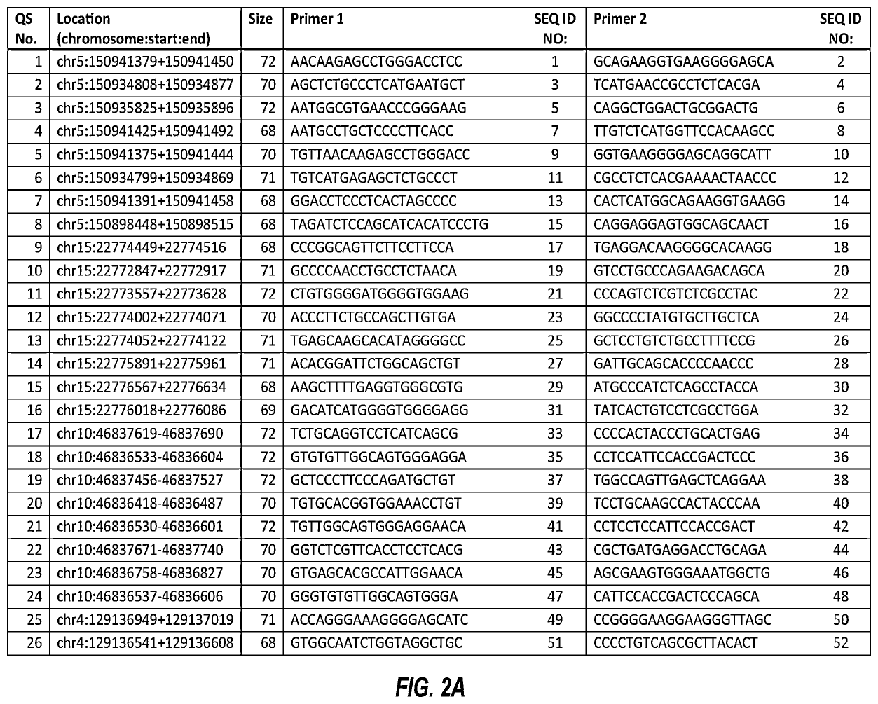

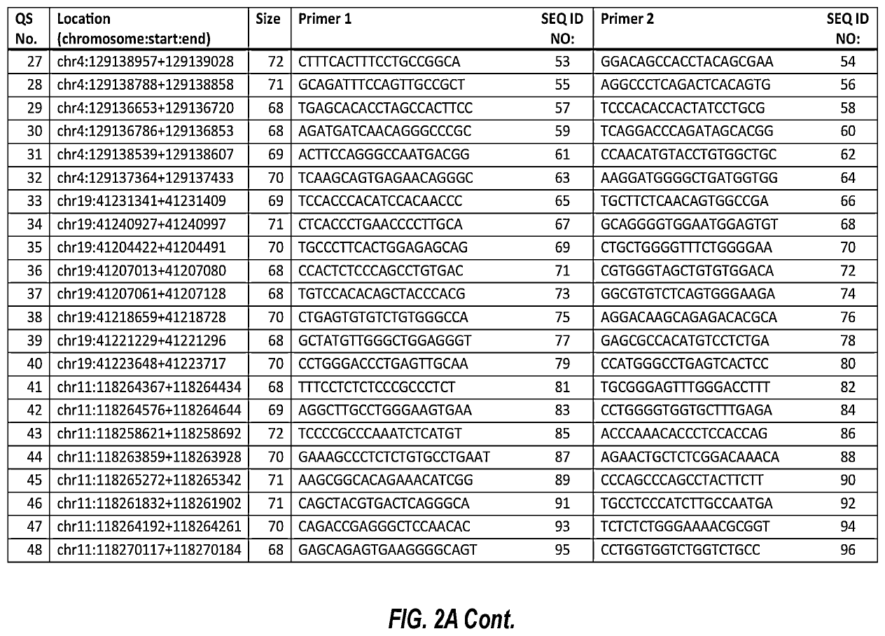

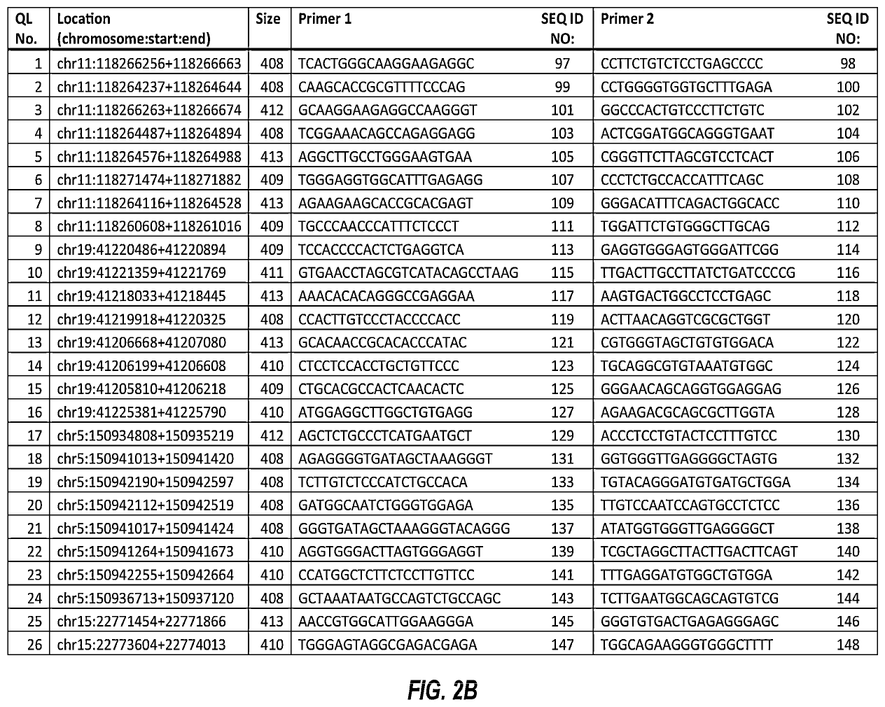

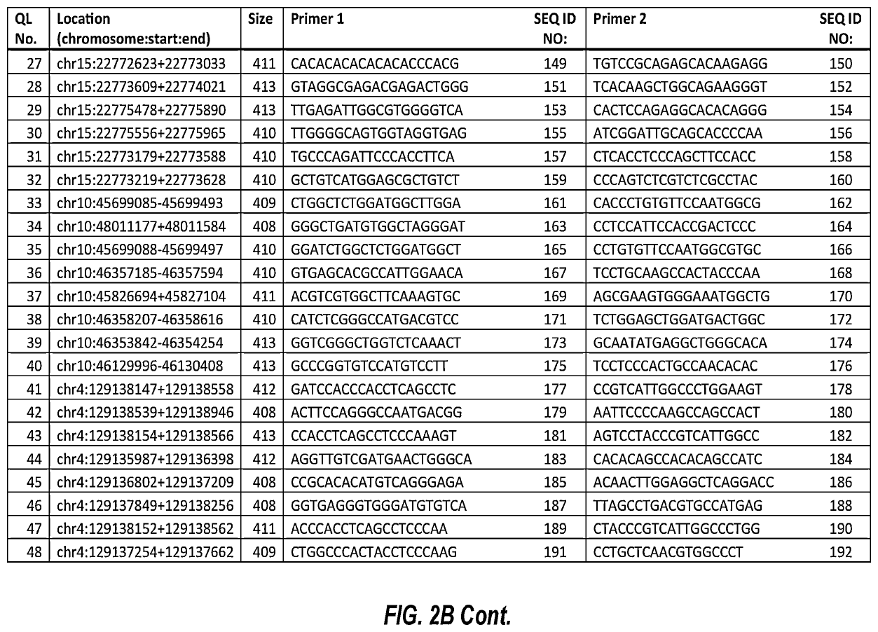

Examples of QS sequences and corresponding QS primers are listed in the table of FIG. 2A. Examples of QL sequences and corresponding QL primers are listed in the table of FIG. 2B. In these Figures, the start and end human genome locations are based on the GenBank Genome Reference Consortium Human Reference 38 (GRCh38; hg38).

### Compositions & Kits for DNA Amplification and Analysis

In some embodiments, the QS and QL standards are included as part of a composition and/or kit for amplifying and analyzing DNA samples. In some embodiments, for example, the composition includes (i) a set of QS primers, (ii) a set of QL primers, (iii) one or more sets of primers each targeted to an STR region of the sample nucleic acid, (iv) optionally, a short internal quality control (IQCS) comprising a synthetic IQCS sequence and a set of primers configured to enable amplification of the IQCS sequence, and (v) optionally, a long internal quality control (IQCL) comprising a synthetic IQCL sequence and a set of primers configured to enable amplification of the IQCL sequence. In some embodiments, the IQCS sequence is shorter than the targeted STR region(s) and the IQCL sequence is longer than the targeted STR region(s). In such embodiments, the IQCS and IQCL bracket the targeted STR regions along the spectrum of analyzed sizes. In other embodiments, the IQCS and/or IQCL sequence(s) have sizes that fall within the range of targeted, variably sized STR loci. In such embodiments, the IQCS and/or IQCL preferably have their own unique size range and/or unique dye label to be sufficiently distinguished from the targeted STR loci on the electropherogram.

The IQCS and IQCL are configured as internal process controls. That is, the IQCS and IQCL can provide information regarding the quality of the testing process, such as whether amplification has been inhibited. However, because the IQCS and IQCL are amplified from their own synthetic template, they cannot function as quantitative standards and cannot provide information specific to the genomic DNA of the sample.

In some embodiments, a kit configured for analysis of nucleic acid samples includes (i) a set of QS primers, (ii) a set of QL primers, (iii) one or more sets of primers each targeted to an STR region of the sample nucleic acid, (iv) optionally, an IQCS, (v) optionally, an IQCL, and (vi) one or more components to enable amplification of target DNA when the kit components are mixed with a sample and subjected to amplification conditions (e.g., thermal cycling to perform polymerase chain reaction (PCR)). The one or more components for enabling amplification may include a lysate, a DNA polymerase, deoxyribonucleotide triphosphate (dNTP) molecules, and one or more buffers and/or salts. In some embodiments, the DNA polymerase is a thermostable DNA polymerase, such as Taq DNA polymerase, a mutant, variant, or derivative thereof. In some embodiments, the dNTP molecules are selected from the group consisting of dTTP, dATP, dCTP, dGTP, 7-deaza-dGTP, or dUTP. Additional or alternative components for enabling amplification of target DNA are known in the art.

### Methods of Amplifying Nucleic Acids & Identifying Individuals

In some embodiments, a method of amplifying one or more nucleic acids within a sample comprises the steps of: (A) providing a sample that includes or is suspected of including nucleic acid; (B) forming a mixture by mixing with the sample a composition comprising (i) at least one set of primers that target an STR region of the sample nucleic acid, (ii) a set of QS primers that target a first QS sequence of the sample nucleic acid, and (iii) a set of QL primers that target a second sequence of the sample nucleic acid, wherein the QS sequence is shorter than the QL sequence; and (C) subjecting the mixture to amplification conditions to enable amplification of targeted nucleic acid if present within the sample.

In some embodiments, the method further comprises identifying an allele of the STR region(s). In some embodiments, the method further comprises identifying an individual based on the determined allele(s).

In some embodiments, a method for identifying a human by analyzing a sample containing nucleic acid from the human comprises the steps of: (A) providing a sample that includes or is suspected as including nucleic acid from the human; (B) forming a mixture by mixing with the sample a composition comprising (i) multiple sets of primers each configured to target a different STR region of the sample nucleic acid, (ii) a set of QS primers that target a QS sequence of the sample nucleic acid, and (iii) a set of QL primers that target a QL sequence of the sample nucleic acid, wherein the QS sequence is shorter than the QL sequence; (C) subjecting the mixture to amplification conditions to enable amplification of targeted nucleic acid within the sample; (D) identifying the alleles of the different STR regions; and (E) identifying the human based on the determined alleles.

In some embodiments, the sample includes or is suspected as including human genomic DNA. In some embodiments, the sample is a forensic sample, crime scene sample, victim sample, or sample from unidentified human remains. The sample may include, for example, blood, bone, buccal material, saliva, semen, urine, feces, skin cells, touch DNA, or a combination thereof.

As discussed above, one limitation common to rapid DNA testing applications is excessive stochastic effects which require the raising of peak height thresholds to compensate. Reducing the number of PCR cycles reduces the number of artifacts present in the resulting STR profile, but also concomitantly reduces the sensitivity of the test. In situations where low levels of sample are available and low levels of DNA are expected (e.g., touch samples, dried blood or other bodily fluid traces on hard surfaces, clothing), the number of PCR cycles is preferably reduced. To compensate for the loss of sensitivity, such methods preferably also utilize flocked swabs for sample collection and/or reduce the relative volume of lysate.

In some embodiments, the sample is collected using a swab. The swab may be a standard cotton swab. Alternatively, in certain embodiments, the swab is a flocked swab. A flocked swab comprises an arrangement of multi-length synthetic fibers attached (e.g., via adhesive) to a substrate/surface. Flocked swabs are preferred in situations where high sensitivity is needed and/or where low levels of sample are available and low levels of DNA are expected. Where larger amounts of sample are available (e.g., buccal swab or sufficient blood), a standard, cotton-tipped swab is acceptable. The volume of sample used as input for amplification is preferably at least about 0.1 μL (e.g., 0.075 μL or greater), or at least about 0.2 μL, or at least about 0.3 μL, or at least about 0.4 μL, or at least about 0.5 μL. The volume of sample used as input is also preferably no greater than about 30 μL, or no greater than about 35 μL, or no greater than about 40 μL. In preferred embodiments, a swab or other sample collection device is configured to absorb and “hold” no more than about 40 μL of the sample and/or no more than about 50% of the available sample.

In some embodiments, the sample is mixed with a lysate before and/or at initiation of amplification. The lysate volume may be varied according to protocol and/or sample amount. In some embodiments, the lysate volume is about 300 μL or more, such as about 500 μL. Alternatively, in certain embodiments, the lysate volume is less than about 300 μL, or less than about 250 μL, or less than about 200 μL, or less than about 150 μL, or about 100 μL. For example, the composition with which the sample is mixed may comprise the lysate at any of the foregoing volumes. Lower lysate volumes are preferred in situations where high sensitivity is needed and/or where low levels of sample are available and low levels of DNA are expected. Where larger amounts of sample are available, the larger lysate volumes (e.g., 300 μL or more) are acceptable.

In some embodiments, the sample is subjected to about 32 cycles of PCR. Alternatively, in certain embodiments, the sample is subjected to less than 32 cycles of PCR, such as 30 cycles. Lower cycle numbers are preferred in situations where high sensitivity is needed and/or where low levels of sample are available and low levels of DNA are expected. Where larger amounts of sample are available, higher cycle numbers (e.g., 32 cycles) are acceptable. In some embodiments, the sample is subjected to more than 32 cycles of PCR. Such embodiments may be implemented where greater sensitivity is desired and results do not suffer from excessive stochastic effects, for example.

### Use of QS and QL Standards to Characterize DNA Profiles

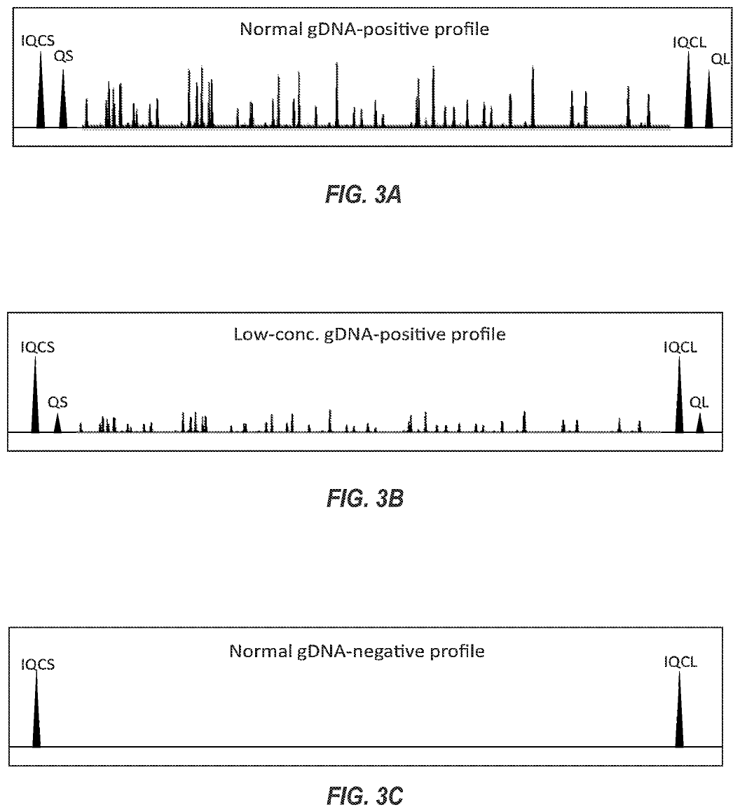

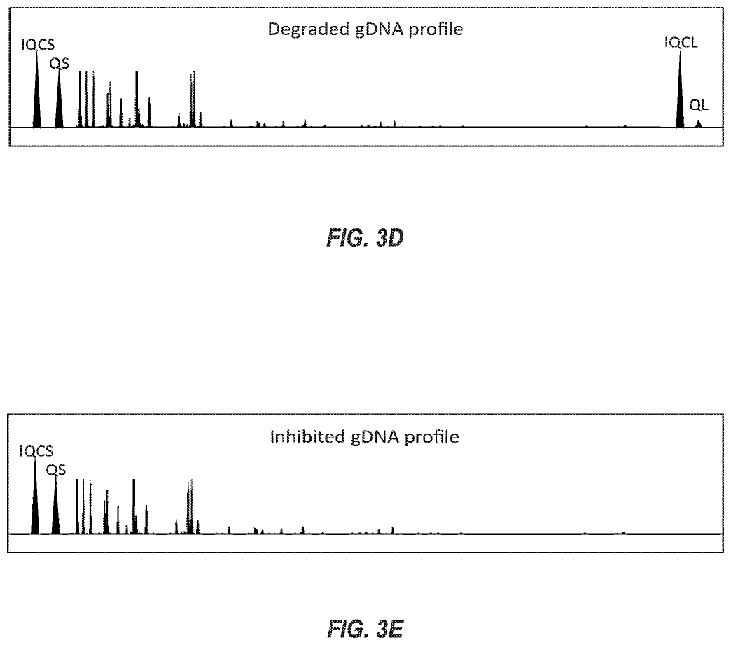

FIGS. 3A-3E illustrate example STR profiles (i.e., electropherograms) for different sample and/or testing scenarios. The STR profiles illustrated here include the QS standard, QL standard, IQCS, and IQCL. In some embodiments, the IQCS and/or IQCL may be omitted.

FIG. 3A illustrates a normal STR profile. As shown, the QS and QL standards are visible and bracket the ends of the STR peaks. This example also includes visible IQCS and IQCL peaks bracketing the ends of the STR peaks. The presence of the IQCS and IQCL peaks indicates that the STR process (including the DNA amplification steps) were carried out effectively. The QS and QL peaks are also substantially similar in height, indicating that the shorter and longer DNA fragments of the sample were similarly amplified.

FIG. 3B illustrates an example STR electropherogram resulting from use of the QS standard, QL standard, IQCS, and IQCL, the results indicating that the level of genomic DNA in the sample is relatively low. As shown, the STR, QS, QL, IQCS, and IQCL peaks are all present, as with the normal profile of FIG. 3A. However, the QS, QL, and STR peaks are lower relative to the profile of FIG. 3A, whereas the IQCS and IQCL peaks remain similar to those of the profile of FIG. 3A. This is because the IQCS and IQCL standards are process controls that utilize their own synthetic templates rather than the genomic DNA of the sample. The size of the IQCS and IQCL peaks therefore does not provide any information about the relative amounts of genomic DNA in the sample. On the other hand, the QS and QL standards are based on primers that target short and long, respectively, regions in the human genome. The QS and QL peaks are therefore proportional to the amount of genomic DNA in the sample and thus to the STR peaks as well.

Peak heights of the QS and/or QL standards can therefore be utilized to quantify the amount of genomic DNA in the sample using, for example, pre-generated standard curves. Such quantification may be utilized to provide a numerical estimate of genomic DNA and/or a categorization of DNA amount such as “high,” “medium,” and “low,” or “stochastic” vs. “non-stochastic.” Typically, the QS is better suited for quantification determinations whereas the QL (and/or a QS to QL ratio) is useful for determining whether the longer STR regions are associated with excessive degradation and/or inhibition effects.

FIG. 3C illustrates an example STR electropherogram resulting from use of the QS standard, QL standard, IQCS, and IQCL, the results indicating negative detection of genomic DNA in the sample. As shown, although the IQCS and IQCL peaks are present, the STR, QS, and QL peaks are absent. The presence of the IQCS and IQCL peaks indicates that the synthetic IQCS and IQCL templates were properly amplified, and thus rules out any overall amplification errors. Had targeted genomic DNA been present in the sample, it would have been expected to result in corresponding QS, QL, and STR peaks. The absence of such peaks therefore indicates that the sample did not include targeted genomic DNA.

FIG. 3D illustrates an example STR electropherogram resulting from use of the QS standard, QL standard, IQCS, and IQCL, the results indicating that genomic DNA in the sample is degraded. As shown, although some STR peaks are present, the peaks show a “ski-slope” profile in which the shorter loci tend to have higher peaks and longer loci have progressively shorter peaks. The IQCS and IQCL are both present. The QS is also present, but the QL is absent or has a height substantially reduced relative to the QS height. This type of profile indicates degraded DNA because longer DNA strands are more likely to be cleaved or otherwise degraded. The IQCL, however, maintains a height similar to the IQCS because it is based on a synthetic template separate from the degraded genomic DNA of the sample.

FIG. 3E illustrates an example STR electropherogram resulting from use of the QS standard, QL standard, IQCS, and IQCL, the results indicating inhibited amplification of genomic DNA in the sample. As shown, some STR peaks are present, but the peaks show a “ski-slope” profile similar to the example of FIG. 3D. unlike the example of FIG. 3D, however, both the QL and the IQCL are reduced in height or absent in this situation. The absence of the IQCL indicates an amplification issue independent of low or degraded genomic DNA in the sample.

In some embodiments, the methods described herein include the steps of determining a first peak height (QS peak height) for a signal associated with the QS sequence and a second peak height (QL peak height) for a signal associated with the QL sequence, determining whether the QS peak height and/or QL peak height fall below respective predetermined thresholds, and flagging the sample as lower quality if the QS peak height and/or QL peak height fall below respective predetermined thresholds. The thresholds may be pre-set by a user, for example.

In some embodiments, when both the QS peak height and the QL peak height fall below their respective predetermined thresholds (e.g., such as in the example of FIG. 3B), the sample is flagged for low nucleic acid concentration. In some embodiments, when the QS peak height and the QL peak height are both substantially zero (e.g., such as in the example of FIG. 3C), the sample is flagged as lacking target nucleic acid.

In some embodiments, when the QS peak height is not lower than its threshold and the QL peak height is lower than its threshold (e.g., such as in the example of FIG. 3D), the sample is flagged as having degraded target nucleic acid and/or as having inhibited amplification. In some embodiments, when the QS peak height is not lower than its threshold, the QL peak height is lower than its threshold, and the IQCL sequence is detected, the sample is flagged as having degraded target nucleic acid. In some embodiments, when the QS peak height is not lower than its threshold, the QL peak height is lower than its threshold, and the IQCL sequence is not detected, the sample is flagged as having inhibited amplification.

In some embodiments, the peak height of the QS standard, the peak height to the QL standard, or both are used to quantify an amount of genomic DNA in the sample. Because the QS and QL sequences from which the QS and QL standards are based are multi-copy sequences within the genomic DNA of the sample, the peak heights can be correlated to the quantity of DNA in the sample. This can be accomplished, for example, by generating a standard curve using known concentrations of DNA and subsequently utilizing the standard curve to calculate the quantity of sample DNA.

### EXAMPLES

### Example 1: Sample Processing Using a Rapid DNA Analysis Instrument

Sample processing was performed using a RapidHIT ID System (Applied Biosystems, Catalog No. A41810). A blood sample was collected using a flocked swab (HydraFlock Swab, available from Puritan Medical Products) and placed in the sample cartridge. The sample cartridge was pre-loaded with assay master mix, primers, and size standard to enable amplification and post-amplification analysis. The sample cartridge was then loaded into the instrument and a small volume (100 μL) lysis protocol was selected. The RapidHIT ID System then performed the sample lysis, DNA amplification, and capillary electrophoresis separation automatically in approximately 90 minutes.

The RapidHIT ID System automatically performed the following steps:
1) Lysis—lysate solution added and lysate mixture heated at 85° C.
2) Bubble Washing—lysate mixture agitated to mix and extract sample DNA from sample matrix.
3) Lysate Pull—lysis volume moved from sample collection chamber to the reaction chamber. A paper punch (1.5 mm diameter) absorbs a portion of the volume while the remaining lysate was flushed into waste chamber.
4) Premix Push—valves opened to allow master mix and primer mix to flood into reaction chamber and immerse the paper punch containing the sample lysate.
5) Thermal Cycling—PCR thermal cycling was set according to Table 3:

TABLE 3
PCR Thermal Cycling

| | | | Duration |
|---|---|---|---|
| Phase | Step | Temperature | (sec) |
| 1: Activation | Hold 1 | 95° C. | 60 |
| | Hold 2 | 61.5° C. | 30 |
| 2: Thermal Cycling | Denature | 94° C. | 3 |
| (repeat 30 cycles) | Anneal/Extend | 61.5° C. | 30 |
| 3: Final Hold | Hold | 60° C. | 480 |

6) Diluent Push—the ILS size standard was mixed with water diluent and pushed into the reaction chamber to transfer the standard, diluent, and PCR reaction components to the mix chamber.
7) Air Pump—cartridge channel between reaction chamber and mix chamber was purged with air.
8) Sample Delivery—Denaturation: Sample mix (PCR reaction+size standard+diluent) passed through tubing in the Denature Heater (95° C.) to denature amplified DNA products and size standard in preparation for CE. Sample DNA injected on CE (5,000 V for 40 kVs total).
9) Electrophoresis and Data Collection—heated CE capillary to 60° C., ramped up voltage to 9,000 V for approximately 25 minutes, collected fluorescent peak data, and transferred electronic data to files in a run folder on a network server.

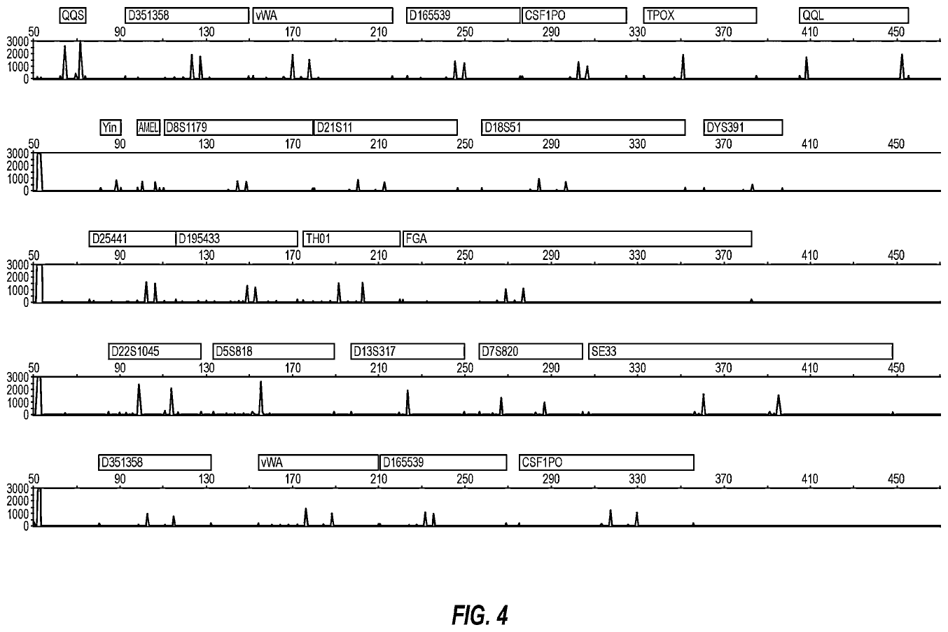

FIG. 4 is a multi-channel electropherogram showing results of the above protocol using Control DNA 007 (see, e.g., Thermo Fisher Scientific, Catalog No. 100028107) and QS and QL markers selected from those shown in FIGS. 2A and 2B. Tested STR markers included D3S1358, vWA, D16S539, CSF1PO, TPOX, Y indel, AMEL, D8S1179, D21S11, D18S51, DYS391, D2S441, D19S433, TH01, FGA, D22S1045, D5S818, D13S317, D7S820, SE33, D10S1248, D1S1656, D12S391, and D2S1338, corresponding to the GlobalFiler™ amplification kit markers.

### Example 2: Blood Volume Peak Height Testing

Using the process as described in Example 1, a blood dilution series was tested to determine the effect of different blood volumes on resulting STR peak heights.

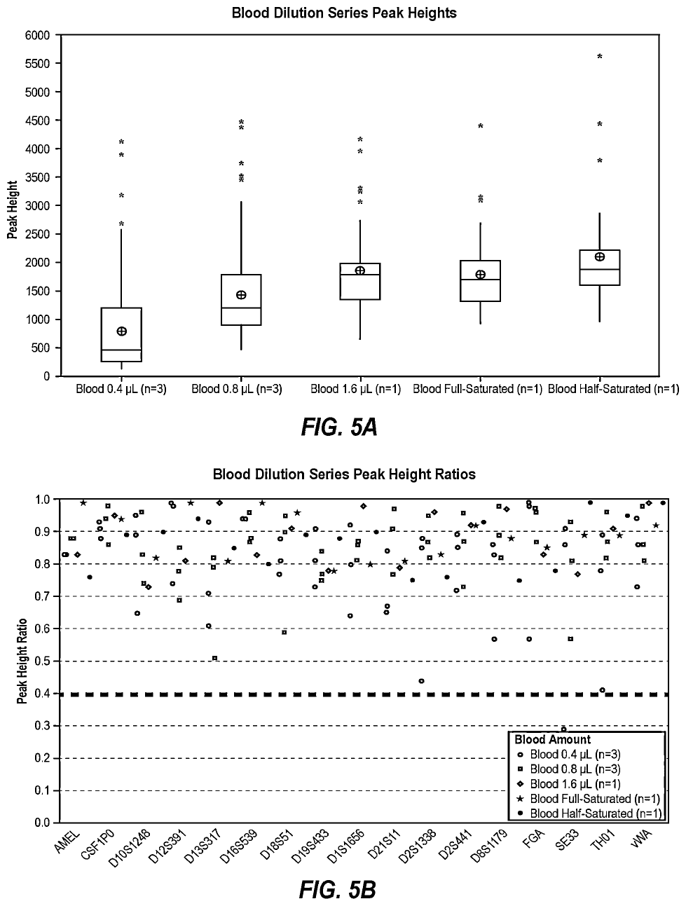

FIG. 5A is a chart illustrating peak heights of STR profiles resulting from a blood dilution series that varied the volume of blood utilized in the rapid DNA analysis, the results showing that suitable peak heights were obtained at blood volumes as low as about 0.4 μL. FIG. 5B is a chart illustrating peak height ratios for different blood volumes at various tested loci, the results showing suitable performance at blood volumes as low as about 0.4 μL.

Additional testing has shown effective results, with minimal allelic dropout, using blood volumes as low as 0.1 μL. Even with some allelic dropout at these low blood inputs, the profile balance is improved relative to conventional rapid investigative leads analysis methods. No allelic dropout was seen when testing blood volumes of 0.2 μL.

### Example 3: Comparison to Conventional Rapid Investigative Leads Analysis

Results from the process as described in Example 1 (1 μL blood input) were compared to results from a conventional rapid DNA process (1 μL blood using a standard cotton swab, 32 PCR cycles, 300 μL lysate).

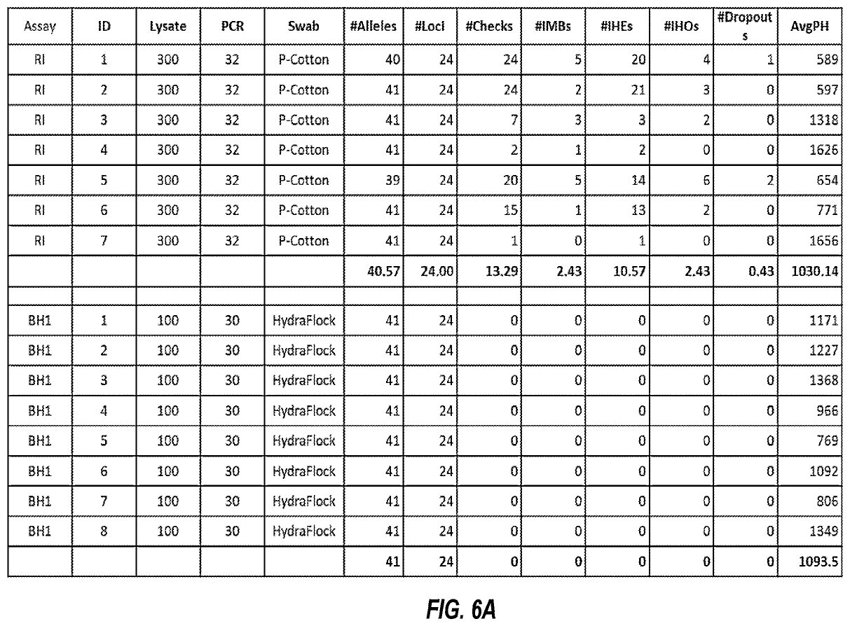

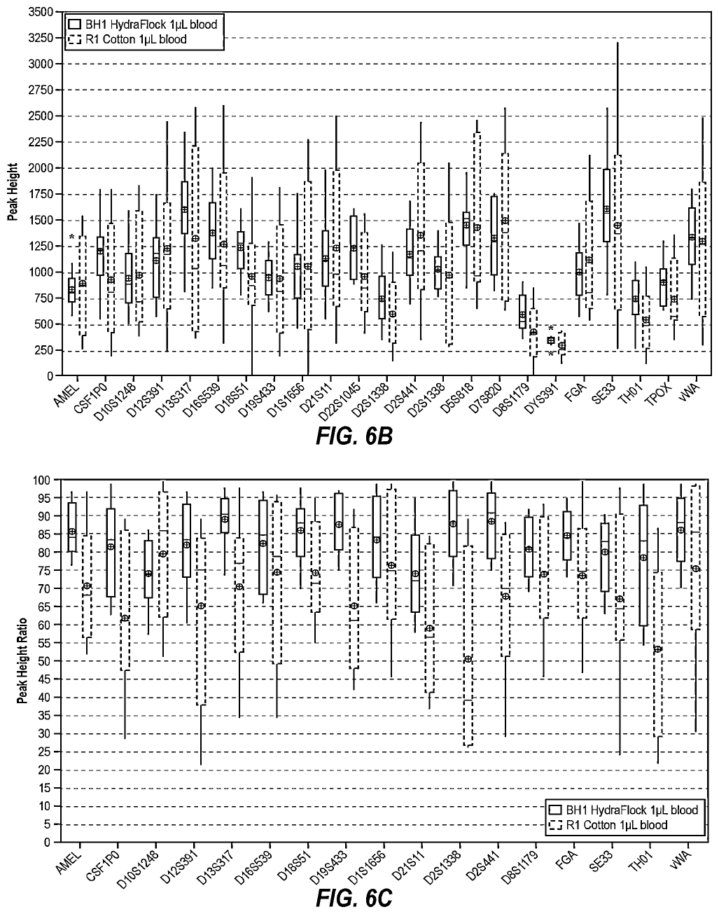

FIG. 6A is a table comparing results of the conventional investigative leads process to an improved process according to the present disclosure, the improved process being modified to use a different collection swab, a lower lysate volume, and lower number of PCR cycles, showing that the modified process reduced the number of flags while also providing effective peak heights.

FIGS. 6B and 6C are charts illustrating peak heights (FIG. 6B) and peak height ratios (FIG. 6C) at various loci, comparing the conventional investigative leads process (“RI Cotton 1 μL blood”) to an improved process according to the present disclosure (“BH1 HydraFlock 1 μL blood”), the results showing that the improved process led to less variance in peak heights.

### Example 4: DNA Input Variation Testing

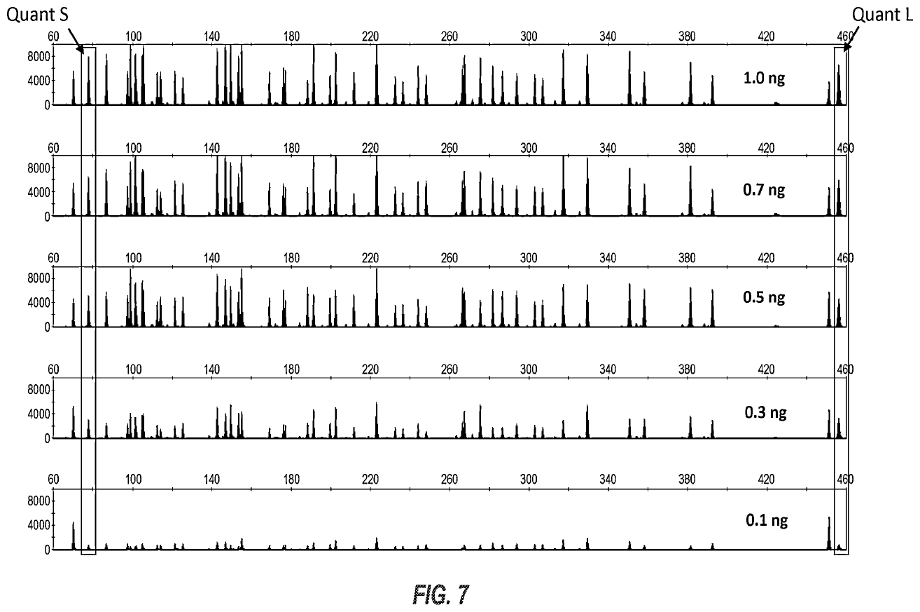

Using the process as described in Example 1 but with the inclusion of QS and QL primers, a series of different input DNA levels (1.0 ng, 0.7 ng, 0.5 ng, 0.3 ng, and 0.1 ng) were tested to determine the effects on QS and QL peaks. FIG. 7 illustrates results of DNA input level testing, showing changes in peaks of the QS standard and QL standard in the corresponding STR profiles at different levels of input DNA. As shown, the QS and QL peak heights were proportional to the level of input DNA.

### Example 5: Quantification Marker Response

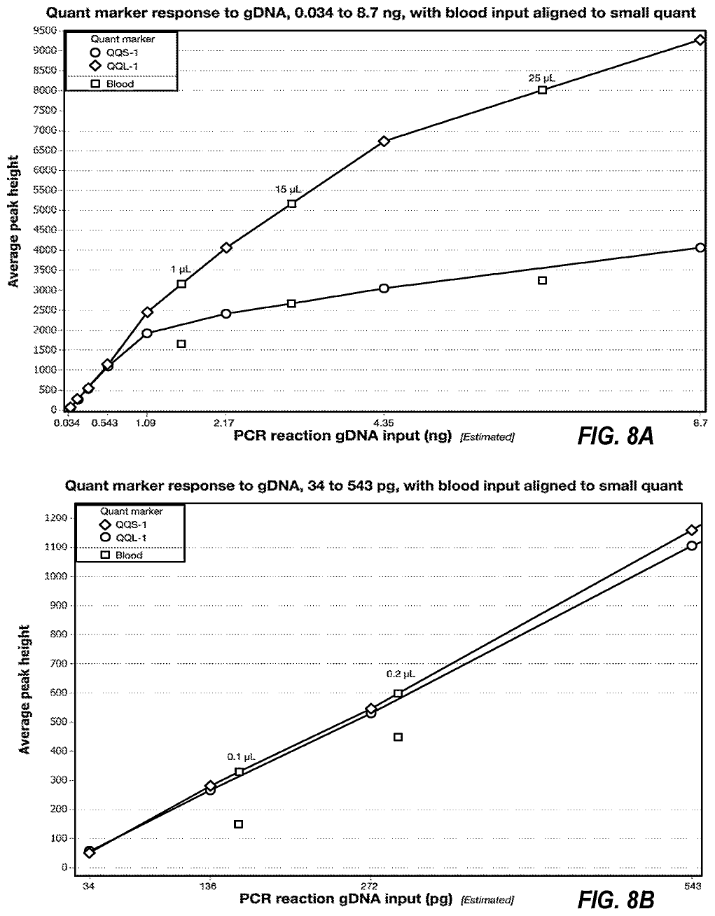

Average peak heights were measured across different levels of gDNA input based on Control DNA 007 (see, e.g., Thermo Fisher Scientific, Catalog No. 100028107) as well as liquid blood at 0.1 μL, 0.2 L, 1 μL, 15 μL, and 25 μL. Results are shown in FIGS. 8A and 8B. FIG. 8A shows the full set of data points (with input DNA shown in ng) and FIG. 8B is an enlarged graph of the first four Control DNA 007 inputs (with input DNA shown in pg). The QS sequences are labelled as “QQS-1”, and the QL sequences are labelled as “QQL-1.” As shown, the peak heights for the blood samples were aligned with the QS curve, which matches well to the estimated PCR DNA capture for each blood volume. Although the QS and QL peak heights were somewhat different, the results are surprisingly effective considering they are roughly 340 base pairs apart from each other.

### Example 6: DNA Degradation Testing

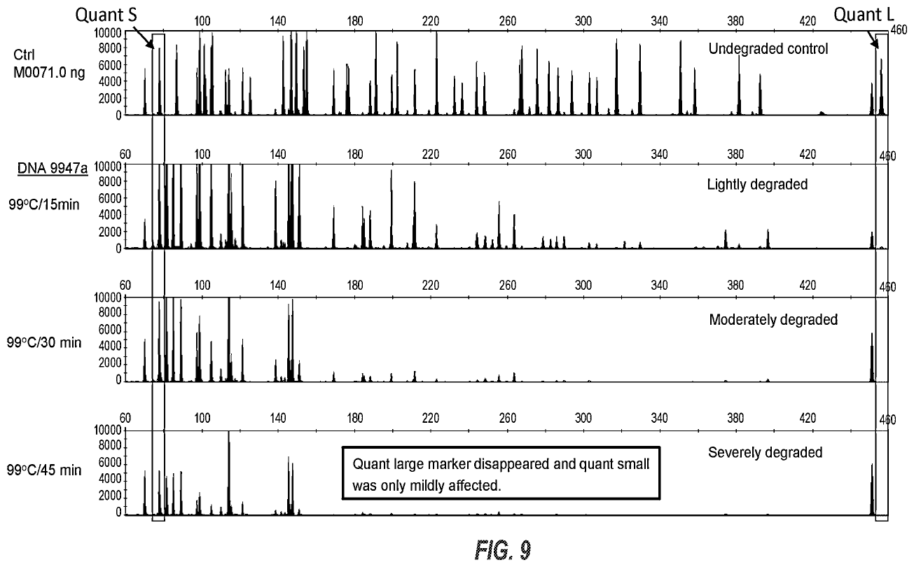

Using the process as described in Example 1 but with the inclusion of QS and QL primers, a series of progressively more degraded samples were tested. FIG. 9 illustrates results of DNA degradation testing, showing greater changes to the QL standard than the QS standard in the corresponding STR profiles at progressively higher levels of DNA degradation.

### Additional Terms & Definitions

Note that this disclosure may alternatively refer to the QS standard as “QTS” or “QQS-QT”, may alternatively refer to the QL standard as “QTL” or “QQL-QT”, may alternatively refer to the IQCS as “QCS” or “QQS-QC”, and may alternatively refer to the IQCL as “QCL” or “QQL-QC.

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. A method for amplifying one or more nucleic acids within a sample, the method comprising:
providing a sample that includes or is suspected of including nucleic acid;
forming a mixture by mixing with the sample a composition comprising:
at least one set of primers that target a short tandem repeat (STR) region of the sample nucleic acid,
a set of short quantification primers (QS primers) that target a first sequence (QS sequence) of the sample nucleic acid, and
a set of long quantification primers (QL primers) that target a second sequence (QL sequence) of the sample nucleic acid, wherein the QS sequence is shorter than the QL sequence; and
subjecting the mixture to amplification conditions to enable amplification of targeted nucleic acid if present within the sample.

2. The method of claim 1, further comprising identifying an allele of the STR region.

3. The method of claim 1, wherein the composition comprises multiple sets of primers each configured to target a different STR region of the sample nucleic acid.

4. The method of claim 3, further comprising identifying the alleles of the different STR regions.

5. The method of claim 1, further comprising identifying an individual based on the determined allele(s).

6. The method of claim 5, wherein identifying the individual is associated with a forensic and/or crime scene investigation.

7. The method of claim 6, wherein the QS sequence is shorter than the STR region(s) and the OL sequence is longer than the STR region(s).

8. (canceled)

9. The method of claim 1, wherein the sample is a forensic sample, crime scene sample, victim sample, or sample from unidentified human remains.

10. The method of claim 1, wherein the sample comprises blood, bone, buccal material, saliva, semen, urine, feces, skin cells, touch DNA, or combination thereof.

11. The method of claim 1, wherein the sample comprises human genomic DNA.

12. The method of claim 1, wherein the QS sequence, the QL sequence, or both are free of indels.

13. The method of claim 1, wherein the QS sequence, the QL sequence, or both are multi-copy sequences.

14. The method of claim 1, wherein at least some of the forward primers and/or at least some of the reverse primers of the set of QS primers and the QL primers include a detectable label.

15. The method of claim 14, wherein the ratio of labelled to non-labelled forward primers in the set of QS primers is about 1:1 to about 1:5, such as about 1:2, and/or wherein the ratio of labelled to non-labelled reverse primers in the set of QS primers is about 1:1 to about 1:5, such as about 1:2.

16. (canceled)

17. The method of claim 16, wherein the ratio of labelled to non-labelled forward primers in the set of QL primers is about 1:1 to about 1:5, such as about 1:2, and/or wherein the ratio of labelled to non-labelled reverse primers in the set of QL primers is about 1:1 to about 1:5, such as about 1:2.

18. The method of claim 1, wherein the set of QS primers is selected from SEQ ID NO:1-SEQ ID NO:96.

19. The method of claim 1, wherein the set of QL primers is selected from SEQ ID NO:97-SEQ ID NO:192.

20. The method of claim 1, further comprising:
determining a first peak height (QS peak height) for a signal associated with the QS sequence and a second peak height (QL peak height) for a signal associated with the QL sequence;
determining whether the QS peak height and/or QL peak height fall below respective predetermined thresholds; and
flagging the sample as lower quality if the QS peak height and/or QL peak height fall below respective predetermined thresholds.

21. The method of claim 20, wherein when both the QS peak height and the QL peak height fall below their respective predetermined thresholds, the sample is flagged for low nucleic acid concentration.

22. The method of claim 20, wherein when the QS peak height is not lower than its threshold and the QL peak height is lower than its threshold, the sample is flagged as having degraded target nucleic acid and/or as having inhibited amplification.

23. The method of claim 20, wherein when the QS peak height and the QL peak height are both substantially zero, the sample is flagged as lacking target nucleic acid.

24. The method of claim 20, wherein the composition further comprises:
a short internal quality control (IQCS) comprising a synthetic IQCS sequence and a set of primers configured to enable amplification of the IQCS sequence; and
a long internal quality control (IQCL) comprising a synthetic IQCL sequence and a set of primers configured to enable amplification of the IQCL sequence,
wherein the IQCS sequence is shorter than the IQCL sequence.

25. The method of claim 24, wherein the IQCS sequence is shorter than the STR region(s) and the IQCL sequence is longer than the STR region(s).

26. (canceled)

27. The method of claim 24, wherein when the QS peak height is not lower than its threshold, the QL peak height is lower than its threshold, and the IQCL sequence is detected, the sample is flagged as having degraded target nucleic acid.

28. The method of claim 24, wherein when the QS peak height is not lower than its threshold, the QL peak height is lower than its threshold, and the IQCL sequence is not detected, the sample is flagged as having inhibited amplification.

29. The method of claim 1, wherein the composition further comprises a DNA polymerase and deoxyribonucleotide triphosphate (dNTP) molecules, and wherein subjecting the mixture to amplification conditions comprises performing a polymerase chain reaction (PCR).

30. The method of claim 29, wherein the DNA polymerase is a thermostable DNA polymerase comprising a Taq DNA polymerase, a mutant, variant, or derivative thereof.

31. The method of claim 30, wherein the DNA polymerase is Taq DNA polymerase, a mutant, variant, or derivative thereof.

32. The method of claim 29, wherein the nucleotides are selected from the group consisting of dTTP, dATP, dCTP, dGTP, 7-deaza-dGTP, or dUTP.

33. (canceled)

34. The method of claim 29, wherein the sample is collected using a non-cotton swab or a flocked swab.

35. (canceled)

36. The method of claim 29, wherein the composition with which the sample is mixed further comprises a lysate, and wherein the composition has a volume of less than about 300 μl before mixing with the sample, or less than about 250 μl before mixing with the sample, or less than about 200 μl before mixing with the sample, or less than about 150 μl before mixing with the sample, or about 100 μl before mixing with the sample.

37. The method of claim 29, wherein the PCR is performed with less than 32 cycles, such as 30 cycles.

38. The method of claim 29, wherein the volume of sample mixed with the composition is at least about 0.1 μl and/or is no greater than about 35 μl.

39. The method of claim 29, further comprising subjecting the amplified nucleic acid to a size separation process.

40. The method of claim 39, wherein the size separation process comprises capillary electrophoresis.

41.-60. (canceled)
